(19) United States
(12) Patent Application Publication
(10) Pub. No.: US 2008/0027072 A1
(43) Pub. Date: Jan. 31, 2008

(54) Potentiation of MC4 receptor activity

(75) Inventors: Teresa A. Bennett, San Diego, CA (US); James R. Hauske, La Jolla, CA (US); Fu-Yue Zeng, Lakeland, TN (US)

Correspondence Address: ROPES & GRAY LLP;PATENT DOCKETING 39/41, ONE INTERNATIONAL PLACE, BOSTON, MA, 02110-2624, US

(73) Assignee: AMPLA Pharmaceuticals, Inc., La Jolla, CA (US)

(21) Appl. No.: 11/788,748

(22) Filed: Apr. 19, 2007

Related U.S. Application Data
(60) Provisional application No. 60/793,843, filed on Apr. 20, 2006.

(51) Int. Cl. A61K 31/496; A61K 31/4523; A61P 15/00; A61P 3/04

(52) U.S. Cl. 514/253.04; 514/323; 514/789

(57) ABSTRACT

The present invention provides methods of treating obesity, eating disorders, and sexual dysfunction. The methods comprise administering to a mammalian host suffering from obesity, an eating disorder, or sexual dysfunction an effective dose of a compound of the invention. Also provided are methods of potentiating the effect of an MC4 receptor agonist in a mammalian host. In some cases the methods comprise administering to the host a compound that lowers the EC₅₀ of the agonist. In other cases the methods comprise administering to the host a compound that increases the maximum effect of the agonist.

### RELATED APPLICATIONS

This application claims the benefit of U.S. Provisional Application No. 60/793,843, filed Apr. 20, 2006, which application is hereby incorporated by reference in its entirety.

### BACKGROUND OF THE INVENTION

Obesity

According to the National Health and Nutrition Examination Survey (NHANES III, 1988 to 1994), between one third and one half of men and women in the United States are overweight. In the United States, sixty percent of men and fifty-one percent of women, of the age of 20 or older, are either overweight or obese. In addition, a large percentage of children in the United States are overweight or obese.

Obesity is a condition of complex origin. Increasing evidence suggests that obesity is not a simple problem of self-control but is a complex disorder involving appetite regulation and energy metabolism. In addition, obesity is associated with a variety of conditions associated with increased morbidity and mortality in a population. Although the etiology of obesity is not definitively established, genetic, metabolic, biochemical, cultural and psychosocial factors are believed to contribute. In general, obesity has been described as a condition in which excess body fat puts an individual at a health risk.

Weight gain and obesity may result from the use of pharmaceutical agents. For example, weight gain is an adverse event associated with the use of virtually all anti-psychotic medications, both traditional and atypical (Arana, J. Clin. Psychiatry 61(Suppl 8):5-13, 2000). The development of obesity in patients taking anti-psychotic agents has been documented since the 1950s. The relative obesity-inducing potential of different atypical anti-psychotics has not been definitively established, although some comparative data are available. Among the atypical agents, clozapine appears to cause the most weight gain; olanzapine, quetiapine, and risperidone an intermediate amount; and ziprasidone the least, although the data on ziprasidone are limited (Taylor et al., Acta Psychiatr. Scand. 101(6):416-32, 2000; Conley et al., J. Clin. Psychiatry 61(Suppl 8):26-30, 2000). Lithium and valproate are considered more likely to cause weight gain than carbamazepine (Zarate, J. Clin. Psychiatry 61(Suppl 8):52-63, 2000). Significant weight gain among patients taking anti-psychotic medications may be a risk factor for non-adherence, especially in young females. Such weight gain also poses increased risk for other obesity-related disorders such as diabetes and heart disease. Dietary strategies and exercise are currently the principal treatments for weight gain among patients using anti-psychotic agents.

There is strong evidence that obesity is associated with increased morbidity and mortality. Disease risk, such as cardiovascular disease risk and type 2 diabetes disease risk, increases independently with increased body mass index (BMI). Indeed, this risk has been quantified as a five percent increase in the risk of cardiac disease for females, and a seven percent increase in the risk of cardiac disease for males, for each point of a BMI greater than 24.9 (Kenchaiah et al., N. Engl. J. Med. 347:305, 2002; Massie, N. Engl. J. Med 347:358, 2002). In addition, there is substantial evidence that weight loss in obese persons reduces important disease risk factors. Even a small weight loss, such as 10% of the initial body weight in both overweight and obese adults has been associated with a decrease in risk factors such as hypertension, hyperlipidemia, and hyperglycemia.

Although diet and exercise provide a simple process to decrease weight gain, overweight and obese individuals often cannot sufficiently control these factors to lose weight effectively. Pharmacotherapy is available; several weight loss drugs have been approved by the Food and Drug Administration that can be used as part of a comprehensive weight loss program. See, e.g., Snow et al., Ann. Intern. Med. 142:525, 2005. However, many of these drugs have serious adverse side effects. When less invasive methods have failed, and the patient is at high risk for obesity-related morbidity or mortality, weight loss surgery is an option in carefully selected patients with clinically severe obesity. However, these treatments are high-risk, and suitable for use in only a limited number of patients. Id.

Pharmacological agents useful in the treatment of obesity may have a variety of targets. For example, U.S. Patent Application Publication No. 2005/0004121, U.S. Pat. No. 6,472,394, and PCT Publication Nos. WO02/04433 and WO2005/019167 report compounds and methods for antagonism of melanin-concentrating hormone in the treatment of obesity and other related disorders.

Other pharmacological agents allegedly useful in the treatment of obesity have been reported. See, e.g., PCT Publication No. WO03/027069 and WO03/040107. Examples of combination therapy in the treatment of obesity and other related disorders have also been described. See, e.g., U.S. Pat. Nos. 5,795,895; 5,908,830; 6,162,805; and 6,548,551.

Eating Disorders

Bulimia nervosa (“ox-like hunger of nervous origin”) was identified as a mental disorder in the early 1970's, but was considered to be an “ominous” variation of the then more recognized eating disorder, anorexia nervosa. Subsequent developments in the study of eating disorders has indicated that, although many anorexia nervosa patients are or may become bulimic, bulimia nervosa is a separate disorder with a distinct set of clinically-defined symptoms and behaviors. The disorder anorexia nervosa can be generally characterized by an individual's refusal to maintain a minimally normal body weight usually effectuated through severe restriction of caloric intake. In contrast, bulimia nervosa and bulimia-related eating disorders are generally characterized by repeated episodes of binge eating, followed by inappropriate and unhealthy compensatory behaviors such as self-induced vomiting; misuse of laxatives, diuretics, or other medications; fasting or excessive exercise.

Bulimia nervosa is of unknown etiology, but it affects a relatively large portion of the population. The Diagnostic and Statistical Manual of Mental Disorders, 4^{th} ed., (DSM-IV), reports the prevalence of bulimia nervosa to be 1% to 3% within the adolescent and young adult female population, and one-tenth of that in the male population. No reliable statistics are available regarding the prevalence of bulimia-type eating disorders in these populations, but it is believed that the rate is similar, or greater, than that of bulimia nervosa. Bulimia nervosa has been reported to occur with roughly similar frequencies in most industrialized countries, including the United States, Canada, Europe, Australia, Japan, New Zealand and South Africa. Thus, within the female population of industrialized nations, bulimia nervosa is at least as common as other major psychiatric disorders such as schizophrenia, which occurs at a rate of 1.5%, and major depressive disorder, which occurs at a rate of 1.3%.

The essential features of bulimia nervosa are a disturbance in perception and a high level of preoccupation with body shape and weight, coupled with binge eating and inappropriate compensatory methods to prevent weight gain. Other characteristic behaviors, as well as the physical and psychological symptoms which give rise to a diagnosis of bulimia nervosa, are well-known in the art and are detailed in the DSM-IV at pages 545 to 550, the contents of which are incorporated herein by reference.

The diagnostic criteria for bulimia nervosa are highly defined; for a diagnosis of bulimia nervosa, individuals must exhibit particular behaviors and psychological symptoms with specified frequency. Frequently individuals engaging in disordered eating practices do not meet these DSM-IV criteria, but exhibit behaviors and thought patterns common to individuals diagnosed with bulimia nervosa, including binge eating, followed by compensatory behaviors and an undue preoccupation with body shape. These individuals are defined by the DSM-IV as having a bulimia-type eating disorder not otherwise specified (“N.O.S.”). The specific clinical criteria defining bulimia-type eating disorder N.O.S. are well-known in the art and are detailed in the DSM-IV at page 550, the contents of which are incorporated herein by reference.

The average age for the onset of bulimia nervosa or bulimia-type eating disorder N.O.S. is late adolescence or early childhood. The overwhelming majority of those who are afflicted, approximately 98%, are young women. In a high percentage of cases, the disturbed eating behavior persists for several years. Recovery rates for bulimia nervosa have been reported at 38% to 46%. The long-term outcome of bulimia nervosa is not known, but anecdotal evidence suggests that relapse is common.

Early epidemiological and family studies of eating disordered individuals demonstrated an apparent linkage between such disorders and mood disturbances. This initial observation has been reinforced further by clinical and physiological data. For example, studies of individuals diagnosed with bulimia nervosa have indicated a high frequency of comorbid diagnoses of axis I psychiatric disorders, including major depressive disorder. Further, research into the pathophysiological bases of eating disorders has implicated a disturbance in the serotonigenic system of eating disordered individuals, a neurotransmitter system also believed to play a role in mood disorders. Because of the several associations of bulimia nervosa and bulimia-type eating disorder N.O.S. with mood and anxiety disorders, most of the treatment modalities devised for bulimia nervosa and bulimia-type eating disorder N.O.S. have been developed from, or have been related to, treatment approaches developed for these disorders. In fact, a brief survey of the scientific literature reveals that, although they are not clinically defined as mood or anxiety disorders, bulimia nervosa and bulimia-type eating disorder N.O.S. are frequently treated with antidepressant medications, such as fluoxetine, imipramine and trazodone. There remains, however, a need for additional methods to treat bulimia nervosa.

Anorexia, defined as the lack or the loss of appetite for food (Dorland's Illustrated Medical Dictionary, 24 edition, W. B. Saunders Company, Philadelphia, 1965) has multiple etiologies. It is commonly associated with cachexia, a state of constitutional disorder, general ill health and malnutrition. Common examples of conditions associated with anorexia and cachexia are anorexia nervosa, certain infectious diseases, and malignancy.

Anorexia nervosa is a serious psychiatric disorder affecting predominantly women (94-96%) in the 13-30 age range. Between 1% (Crisp et al., 128 Br. J. Psychiatry 549, 1976) and 3% (Ballot et al., 59 S. Afr. Med. J. 992, 1981) of young women may be affected. The morbidity and mortality from this condition are considerable. Two years from diagnosis, 4-6% have died and only 50% have achieved a normal weight. There are multiple endocrine and metabolic abnormalities present, most of which are believed to be secondary to the malnutrition. A serious complication of the condition is osteoporosis, which can involve both the spine and peripheral bones. At present there is no specific treatment for anorexia nervosa, although multiple approaches have been tried (Piazza et al., Compr. Psychiatry 21:177-189 1980). Improved treatments for anorexia are therefore needed.

Sexual Dysfunction

Sexual difficulties can begin early in a person's sex life or they may develop after an individual has previously experienced enjoyable and satisfying sex. A problem may develop gradually over time, or may occur suddenly as a total or partial inability to participate in one or more stages of the sexual act. The causes of sexual difficulties can be physical, psychological, or both.

Emotional factors affecting sex include both interpersonal problems and psychological problems within the individual. Physical factors include drugs, injuries to the back, problems with an enlarged prostate gland, problems with blood supply, nerve damage, failure of various organ systems, endocrine disorders, hormonal deficiencies, and some birth defects.

Sexual dysfunction disorders are generally classified into four categories: sexual desire disorders, sexual arousal disorders, orgasm disorders, and sexual pain disorders. Sexual desire disorders or decreased libido can be caused by a decrease in normal estrogen (in women) or testosterone (in both men and women) production. Other causes may be aging, fatigue, pregnancy, medications (such as the SSRIs) or psychiatric conditions, such as depression and anxiety. Sexual arousal disorders were previously known as frigidity in women and impotence in men, though these have now been replaced with less judgmental terms. Impotence is now known as erectile dysfunction, and frigidity has been replaced with a number of terms describing specific problems with, for example, desire or arousal. For both men and women, these conditions can manifest as an aversion to, and avoidance of, sexual contact with a partner. In men, there may be partial or complete failure to attain or maintain an erection, or a lack of sexual excitement and pleasure in sexual activity. There may be medical causes to these disorders, such as decreased blood flow or lack of vaginal lubrication. Chronic disease can also contribute, as well as the nature of the relationship between the partners. As is now clear from the recent success of pharmacological therapy for erectile dysfunction, most erectile disorders in men are primarily physical, not psychological conditions.

Orgasm disorders are a persistent delay or absence of orgasm following a normal sexual excitement phase. The disorder can occur in both women and men. Again, the SSRI antidepressants are frequent culprits—these can delay the achievement of orgasm or eliminate it entirely.

Sexual pain disorders affect women almost exclusively and are known as dyspareunia (painful intercourse) and vaginismus (an involuntary spasm of the muscles of the vaginal wall that interferes with intercourse). Dyspareunia may be caused by insufficient lubrication (vaginal dryness) in women. Poor lubrication may result from insufficient excitement and stimulation, or from hormonal changes caused by menopause, pregnancy, or breast-feeding. Irritation from contraceptive creams and foams can also cause dryness, as can fear and anxiety about sex.

Although pharmacological therapies for sexual dysfunction are becoming more common, there is clearly a need for additional approaches.

Melanocortins and Melanocortin Receptors

Melanocortins are a group of pituitary peptide hormones that include adrenocorticotropin (“ACTH”) and the α-, β-, and γ-melanocyte stimulating hormones (“MSH”). The melanocortins are known to affect adrenal cortical function and melanocytes. In particular, the melanocortin system is implicated in the regulation of learning and grooming behaviors. Melanocortins are also known to exhibit a variety of cardiovascular effects. See Voisey et al., Curr. Drug Targets 4:586, 2003.

The melanocortin receptors belong to the G-protein coupled receptor superfamily. At least five melanocortin receptors have been cloned: MC1-R, MC2-R, MC3-R, MC4-R, and MC5-R. The receptors are coupled primarily through adenylate cyclase, although other pathways may also be involved in signal transduction. The receptors are reportedly involved in pigmentation, inflammation, steroidogenesis, energy homeostasis, sexual behavior, appetite regulation, and exocrine function. Id.

The MC4 receptor is a 333-residue protein that is expressed primarily in the brain. Gantz et al., J. Biol. Chem. 268:15174, 1993. Agonists of the MC4 receptor include ACTH, α-MSH, and β-MSH. Id. Antagonists include the agouti protein and agouti-related protein. Lu et al., Nature 371:799, 1994; Ollmann et al., Science 278:135, 1997. Ectopic expression of agouti or targeted disruption of the MC4 receptor result in obesity in mice, demonstrating a brain signaling pathway that controls nutrient intake and energy balance. Huszar et al., Cell 88:131, 1997. Synthetic agonists of the MC3 and MC4 receptors inhibit feeding in mice in a dose-dependent manner, and a synthetic, agouti-mimetic, antagonist of the receptors enhances feeding in mice. Fan et al., Nature 385:165, 1997. Tissue-specific and agonist-specific differentiation of the various melanocortin receptors is necessary to characterize the relative physiological roles of each receptor. For example, a selective, non-peptide agonist of the MC4 receptor reportedly augments erectile activity initiated by electrical stimulation of the cavernous nerve in a receptor-dependent manner. Van der Ploeg et al., Proc. Nat'l Acad. Sci. U.S.A. 99:11381, 2002. The agonist also reportedly enhances copulatory behavior in mice. Id.

The MC4 receptor has been targeted in methods of screening compounds that may regulate body weight. U.S. Pat. No. 5,908,609. Methods of treating obesity and other disorders using compounds that attenuate the binding of agouti-related protein to melanocortin receptors but that do not attenuate the binding of α-MSH to the receptors have also been proposed. U.S. Pat. Nos. 6,451,783 and 6,734,175. Agonists of MC4 allegedly useful for the treatment of obesity, diabetes, and male and/or female sexual dysfunction have been reported. U.S. Pat. Nos. 6,376,509; 6,350,760; 6,458,790; and 6,818,658. Despite these reports, however, no effective methods of treating obesity, other eating disorders, or sexual dysfunction by potentiation of a melanocortin receptor agonist are currently known. Thus, a need remains for agents and methods for such treatment.

### SUMMARY OF THE INVENTION

The present invention solves these and other problems by providing methods of treating obesity, other eating disorders, and sexual dysfunction. Also provided are methods of potentiating the effect of an MC4 receptor agonist and/or attenuating the effect of an MC4 receptor inverse agonist in a mammalian host. In certain embodiments, the methods comprise allosterically potentiating the effect of an MC4 receptor agonist and/or allosterically attenuating the effect of an MC4 receptor inverse agonist in a mammalian host. In one aspect, the methods comprise administering to the mammalian host an effective anti-obesity dose of a compound of any one of structural formulae I to IV or a pharmaceutically acceptable salt thereof, or a solvate or prodrug of the compound or its salt: 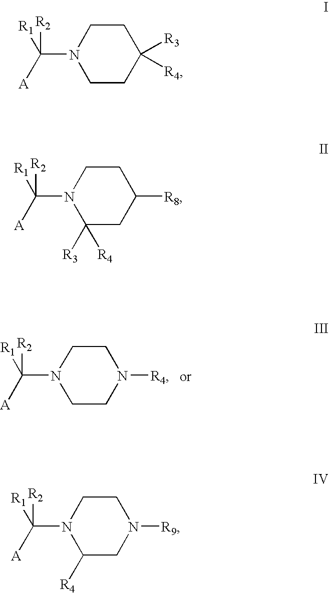
wherein:
R₁ and R₂ are each H or taken together are ═O;
R₃ is selected from OH, halogen, acyl, or substituted or unsubstituted C₁₋₆alkyl;
R₄ is selected from substituted or unsubstituted phenyl, substituted or unsubstituted thiophene, or ring B, wherein B is

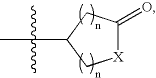

and ring B comprises 5, 6, or 7 atoms in the ring;

X is selected from O or NR₁₀;

R₁₀ is selected from H or substituted or unsubstituted C₁₋₆alkyl;

n is independently for each occurrence selected from 0, 1, 2, 3, or 4;

R₈ is selected from H, OH or substituted or unsubstituted C₁₋₆alkyl;

R₉ is selected from H or substituted or unsubstituted C₁₋₆alkyl;

A is selected from 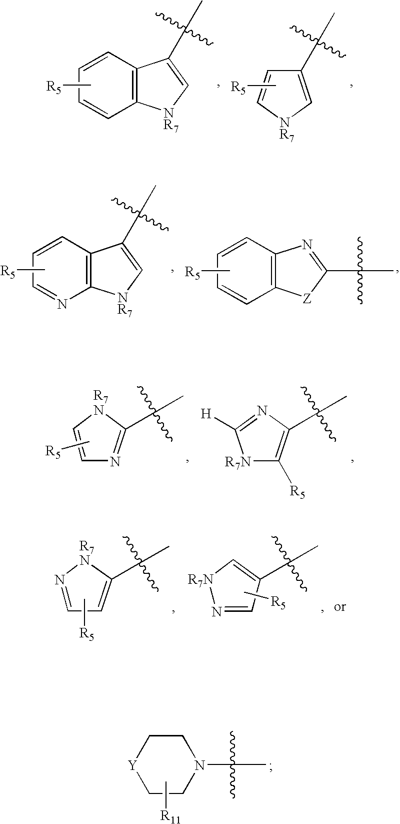
R₅ is independently for each occurrence selected from H, substituted or unsubstituted alkyl, alkenyl, alkynyl, aryl, aralkyl, alkoxy, aryloxy, aralkoxy, cycloalkyl, cycloalkoxy, heterocyclyl, heterocyclyloxy, heterocyclylalkyl, heteroaryl, or heteroaralkyl, keto, hydroxy, substituted or unsubstituted alkylthio, amino, alkylamino, alkanoylamino, aroylamino, or aralkanoylamino, carboxy, substituted or unsubstituted carboxyalkyl, carboxamidoalkyl, thiocarboxy, thiocarboxyalkyl, or thiocarboxamidoalkyl, halo, cyano, nitro, formyl, acyl, or substituted or unsubstituted sulfonyl, or sulfonamido, and is optionally substituted with 1-3 R₆ groups;
R₁₁ is independently for each occurrence selected from H, substituted or unsubstituted alkyl, alkenyl, alkynyl, aryl, aralkyl, alkoxy, aryloxy, aralkoxy, cycloalkyl, cycloalkoxy, heterocyclyl, heterocyclyloxy, heterocyclylalkyl, heteroaryl, or heteroaralkyl, keto, hydroxy, substituted or unsubstituted alkylthio, amino, alkylamino, alkanoylamino, aroylamino, or aralkanoylamino, carboxy, substituted or unsubstituted carboxyalkyl, carboxamidoalkyl, thiocarboxy, thiocarboxyalkyl, or thiocarboxamidoalkyl, halo, cyano, nitro, formyl, acyl, or substituted or unsubstituted sulfonyl, or sulfonamido, and is optionally substituted with 1-3 R₆ groups;
R₆ is independently for each occurrence selected from substituted or unsubstituted alkyl, aryl, aralkyl, alkoxy, aryloxy, heterocyclyl, or heterocyclyloxy, keto, hydroxy, substituted or unsubstituted alkylthio, amino, alkanoylamino, or aroylamino, carboxy, substituted or unsubstituted carboxyalkyl, or carboxamidoalkyl, halo, cyano, nitro, formyl, or substituted or unsubstituted sulfonyl, or sulfonamido;
Y is CH₂, O, S or NR₁₀;
Z is selected from O or NR₇; and
R₇ is independently for each occurrence selected from H or substituted or unsubstituted C₁₋₁₆alkyl.

In some embodiments of the invention, R₄ is 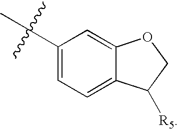
In other embodiments, R₄ is 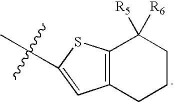
In still other embodiments, the compound is enriched in a particular stereoisomer.

In another aspect, the invention provides methods of potentiating the effect of an MC4 receptor agonist in a mammalian host. In some embodiments, the methods comprise administering to the mammalian host a compound that lowers the EC₅₀ of the agonist for the MC4 receptor. In other embodiments, the methods comprise administering to the mammalian host a compound that increases the maximum effect of the agonist on the MC4 receptor. In some embodiments, the compound is an allosteric potentiator of the MC4 receptor agonist. In some embodiments, the agonist is α-MSH or NDP α-MSH. In some embodiments, the compound is administered in an effective dose. In some embodiments, the compound is a compound of the invention (e.g., a compound of any one of formulae I to IV or a pharmaceutically acceptable salt thereof, or a solvate or prodrug of the compound or its salt).

In another aspect, the invention provides methods of attenuating the effect of an MC4 receptor inverse agonist in a mammalian host. In some embodiments, the methods comprise administering to the mammalian host a compound that raises the EC₅₀ of an inverse agonist for the MC4 receptor. In other embodiments, the methods comprise administering to the mammalian host a compound that decreases the maximum effect of an inverse agonist on the MC4 receptor. In some embodiments, the compound is an allosteric attenuator of a MC4 receptor inverse agonist, such as agouti-related peptide (AgRP). In some embodiments, the compound is administered in an effective dose. In some embodiments, the compound is a compound of the invention (e.g., a compound of any one of formulae I to IV or a pharmaceutically acceptable salt thereof, or a solvate or prodrug of the compound or its salt).

In another aspect, the invention provides methods of treating eating disorders comprising administering to a mammalian host an effective dose of a compound of the invention (e.g., a compound of any one of formulae I to IV or a pharmaceutically acceptable salt thereof, or a solvate or prodrug of the compound or its salt). In some embodiments, the eating disorder is bulimia nervosa or bulimia-type eating disorder not otherwise specified. In other embodiments, the eating disorder is anorexia nervosa.

In still another aspect, the invention provides methods of treating sexual dysfunction comprising administering to a mammalian host an effective dose of a compound of the invention (e.g., a compound of any one of formulae I to IV or a pharmaceutically acceptable salt thereof, or a solvate or prodrug of the compound or its salt). The sexual dysfunction treated according to some embodiments of the invention is male sexual dysfunction. In other embodiments, the sexual dysfunction is female sexual dysfunction. In certain specific embodiments, the sexual dysfunction is erectile dysfunction.

The invention also encompasses methods of treating obesity, eating disorders, and sexual dysfunction, and methods of potentiating the effect of an MC4 receptor agonist in a mammalian host, wherein a compound, such as a compound of the invention (e.g., a compound of any one of formulae I to IV or a pharmaceutically acceptable salt thereof, or a solvate or prodrug of the compound or its salt), is administered as a pharmaceutical composition comprising a pharmaceutically acceptable carrier. In some embodiments, the compound is an allosteric potentiator of the MC4 receptor agonist. In some embodiments, the methods of the invention further comprise administering to the mammalian host an antagonist of the CB1 receptor, an agonist of the MC4 receptor, an inhibitor of dopamine reuptake, an inhibitor of norepinephrine reuptake, an inhibitor of both dopamine and norepinephrine reuptake, or a dopamine agonist or partial agonist.

In another aspect, the present invention provides a method of treating obesity, eating disorders, and sexual dysfunction, or a method of potentiating the effect of an MC4 receptor agonist in a patient in need of anti-psychotic treatment, comprising administering to said patient a compound of the invention (e.g., a compound of any one of formulae I to IV or a pharmaceutically acceptable salt thereof, or a solvate or prodrug of the compound or its salt). In another aspect, the present invention provides a method of treating obesity, eating disorders, and sexual dysfunction, or a method of potentiating the effect of an MC4 receptor agonist in a patient being treated with one or more anti-psychotic agents, comprising administering to said patient a compound of the invention (e.g., a compound of any one of formulae I to IV or a pharmaceutically acceptable salt thereof, or a solvate or prodrug of the compound or its salt). In some embodiments, the compound is an allosteric potentiator of the MC4 receptor agonist.

The details of various aspects of the invention are set forth in the accompanying drawings and the description below. Other features, objects, and advantages of the invention will be apparent from the description and claims.

### LISTING OF DRAWINGS

FIG. 1. (A) Dot plots of cells loaded with Indo-1. (B) Dot plate displaying cells as run through the Direct Sample Injection System.

FIG. 2. α-MSH dose-response curve in the absence and presence of 1 μM Compound 1.

FIG. 3. α-MSH dose-response curve in the presence of several different concentrations of Compound 1.

FIG. 4. Dissociation kinetics assay.

FIG. 5. Binding competition assay.

FIG. 6. Binding competition assay.

FIG. 7. ELISA-based cAMP assay.

FIG. 8. ELISA-based cAMP assay.

FIG. 9. α-MSH dose-response in single-well kinetics (a); α-MSH dose-response in single-well kinetics with 300 nM of Compound 1 (b); α-MSH dose-response in single-well kinetics with 1 μM of Compound 1 (c); α-MSH dose-response in single-well kinetics with 3 μM of Compound 1 (d); α-MSH dose-response in single-well kinetics with 10 μM of Compound 1 (e).

FIG. 10. Measurement of food intake as compared to vehicle for MC4 knockout mice (b) and wild-type (a).

FIG. 11. Measurement of body weight as compared to vehicle for MC4 knockout mice (b) and wild-type (a).

FIG. 12. Measurement of the effect of Treatment X, Sibutramine, and Rimonabant on the body weight of mice which exhibit diet-induced obesity.

FIG. 13. Measurement of the effect of Treatment X, Sibutramine, and Rimonabant on the mean daily food intake of mice which exhibit diet-induced obesity.

### DETAILED DESCRIPTION OF THE INVENTION

The present invention relates to methods of treating obesity, eating disorders, and sexual dysfunction, as well as methods of potentiating or attenuating the effect of an MC4 ligand. In particular, the invention relates to allosteric potentiators of the MC4 receptor. The invention further relates to allosteric attenuators of the MC4 receptor. Among the compounds useful in the methods of the present invention are piperidine and piperazine derivatives.

Before further description of the invention, certain terms employed in the specification, examples and appended claims are, for convenience, collected here.

The term “allosteric attenuator” as used herein, refers to a compound that binds to the MC4 receptor in the presence of an inverse agonist and decreases the maximum effect or raises the EC₅₀ of the inverse agonist. As it relates to the present invention, an “allosteric attenuator” does not agonize, antagonize, or act as an inverse agonist on the MC4 receptor in the absence of the endogenous inverse agonist.

The term “allosteric potentiator” as used herein, refers to a compound that binds to the MC4 receptor in the presence of an agonist and increases the maximum effect or lowers the EC₅₀ of the agonist. As it relates to the present invention, an “allosteric potentiator” does not agonize, antagonize, or act as an inverse agonist on the MC4 receptor in the absence of the endogenous agonist.

The term “hydrate” as used herein, refers to a compound formed by the association of water with the parent compound.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “solvate” as used herein, refers to a compound formed by solvation (e.g., a compound formed by the combination of solvent molecules with molecules or ions of the solute).

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “acyl” is art-recognized and refers to a group represented by the general formula hydrocarbylC(O)—, in certain specific embodiments, alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, in certain specific embodiments, alkylC(O)O—.

The term “alkoxy” refers to an alkyl group, in certain specific embodiments, a lower alkyl group, having an oxygen attached thereto. Representative alkoxy groups include methoxy, ethoxy, propoxy, tert-butoxy, and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In some embodiments, a straight chain or branched chain alkyl has 30 or fewer carbon atoms in its backbone (e.g., C₁-C₃₀ for straight chains, C₃-C₃₀ for branched chains), and more specifically 20 or fewer. Likewise, some cycloalkyls have from 3-10 carbon atoms in their ring structure, and more specifically have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substitutents on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀alkyl indicates a hydrogen where the group is in a terminal position, or is a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond, respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group 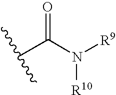
wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by 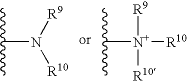
wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. In certain embodiments, the ring is a 5- to 7-membered ring, and in more specific embodiments is a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group 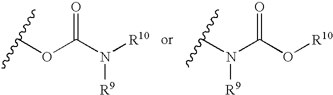
wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, “carbocyclyl”, and “carbocyclic”, as used herein, refer to a non-aromatic saturated or unsaturated ring in which each atom of the ring is carbon. In certain embodiments, a carbocycle ring contains from 3 to 10 atoms, and in more specific embodiments from 5 to 7 atoms.

The term “carbocyclylalkyl”, as used herein, refers to an alkyl group substituted with a carbocycle group.

The term “carbonate” is art-recognized and refers to a group —OCO₂—R⁹, wherein R⁹ represents a hydrocarbyl group.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein mean halogen and include chloro, fluoro, bromo, and iodo.

The terms “hetaralkyl” and “heteroaralkyl”, as used herein, refer to an alkyl group substituted with a hetaryl group.

The terms “heteroaryl” and “hetaryl” include substituted or unsubstituted aromatic single ring structures, in certain specific embodiments 5- to 7-membered rings, more specifically 5- to 6-membered rings, whose ring structures include at least one heteroatom, in some embodiments one to four heteroatoms, and in more specific embodiments one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Typical heteroatoms are nitrogen, oxygen, and sulfur.

The terms “heterocyclyl”, “heterocycle”, and “heterocyclic” refer to substituted or unsubstituted non-aromatic ring structures, in certain specific embodiments 3- to 10-membered rings, more specifically 3- to 7-membered rings, whose ring structures include at least one heteroatom, in some embodiments one to four heteroatoms, and in more specific embodiments one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, and in certain embodiments, six or fewer. A “lower alkyl”, for example, refers to an alkyl group that contains ten or fewer carbon atoms, in specific embodiments six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, more specifically from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc., under normal conditions. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically described as “unsubstituted”, references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae 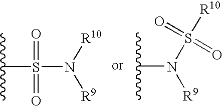
wherein R⁹ and R¹⁰ independently represents hydrogen or hydrocarbyl, such as an alkyl group, or R⁹ and R¹⁰ taken together with the intervening atom(s) complete a heterocycle having from 4 to 8 atoms in the ring structure.

The term “sulfoxide” is art-recognized and refers to the group —S(O)—R⁹, wherein R⁹ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R⁹, wherein R⁹ represents a hydrocarbyl.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula 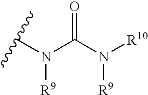
wherein R⁹ and R¹⁰ independently represent hydrogen or a hydrocarbyl, such as an alkyl group, or either occurrence of R⁹ taken together with R¹⁰ and the intervening atom(s) complete a heterocycle having from 4 to 8 atoms in the ring structure.
Methods of Treating Obesity

The present invention relates to methods of treating obesity in a mammalian host. In one aspect, the methods comprise administering to a mammalian host suffering from obesity an effective anti-obesity dose of a compound of the invention (e.g., a compound of any one of formulae I to IV or a pharmaceutically acceptable salt thereof, or a solvate or prodrug of the compound or its salt)

In preferred embodiments of the methods of the invention, the mammalian host is a human.

As used herein, the term “obesity” includes both excess body weight and excess adipose tissue mass in a mammal. In some embodiments of the invention, obesity includes body weight or adipose tissue mass that is in excess of that considered desirable by the individual mammal. In other embodiments, obesity includes body weight or adipose tissue mass considered unhealthy by a physician or veterinarian. In some embodiments, obesity may be assessed quantitatively, for example, by calculation of an individual's body mass index (“BMI”), where
BMI=Weight (in kg.)/(Height (in meters))².

See, e.g., Flier and Maratos-Flier, Harrison's Principles of Internal Medicine, Ch. 64, McGraw-Hill, 2004. Therapeutically-effective anti-obesity dosages are determined according to standard methods. See below. In certain specific embodiments, an individual human mammal may be considered obese if the individual's BMI is at least 20, at least 25, at least 30, at least 35, at least 40, at least 45, or even higher. The treatment of obesity, as provided in methods of the present invention, contemplates not only the treatment of individuals who are defined as “obese”, but also the treatment of individuals with weight gain that if left untreated may lead to the development of obesity.

Methods of Treating Eating Disorders

The present invention also relates to methods of treating eating disorders in a mammalian host. In one aspect, the methods comprise administering to a mammalian host suffering from an eating disorder an effective dose of a compound of the invention (e.g., a compound of any one of formulae I to IV or a pharmaceutically acceptable salt thereof, or a solvate or prodrug of the compound or its salt). In some embodiments, the eating disorder is bulimia nervosa or bulimia-type eating disorder not otherwise specified. In other embodiments, the eating disorder is anorexia nervosa. See, e.g., Walsh, Harrison's Principles of Internal Medicine, Ch. 65, McGraw-Hill, 2004. Therapeutically-effective dosages and pharmaceutical compositions are further described below.

In preferred embodiments of the methods of the invention, the mammalian host is a human.

Methods of Treating Sexual Dysfunction

In yet another aspect, the present invention relates to methods of treating sexual dysfunction in a mammalian host. Accordingly, the methods comprise administering to a mammalian host suffering from sexual dysfunction an effective dose of a compound of the invention (e.g., a compound of any one of formulae I to IV or a pharmaceutically acceptable salt thereof, or a solvate or prodrug of the compound or its salt). Male and female sexual dysfunction are diagnosed and characterized according to standard clinical practice. See, e.g., McVary, Harrison's Principles of Internal Medicine, Ch. 43, McGraw-Hill, 2004. Administration of an appropriate, therapeutically-effective amount of a compound of the invention (e.g., a compound of any one of formulae I to IV or a pharmaceutically acceptable salt thereof, or a solvate or prodrug of the compound or its salt) is likewise well understood by those of skill in the art. See below. The compounds may be administered as pharmaceutical compositions, and they may also be administered in combination with other pharmacological therapies useful in the treatment of sexual dysfunction. See, id. Examples of compounds known to be effective in such treatment include phosphodiesterase inhibitors (see, e.g., U.S. Pat. Nos. 5,250,534; 5,859,006; 6,140,329; 6,362,178; 6,469,012; 6,821,975; and 6,943,166) and dopamine receptor agonists (see, e.g., PCT International Publication No. WO03/051370).

In preferred embodiments of the methods of the invention, the mammalian host is a human.

Compounds of the Invention

Compounds suitable for use in methods of the invention include compounds represented by any one of structural formulae I to IV or a pharmaceutically acceptable salt thereof, or a solvate or prodrug of the compound or its salt: 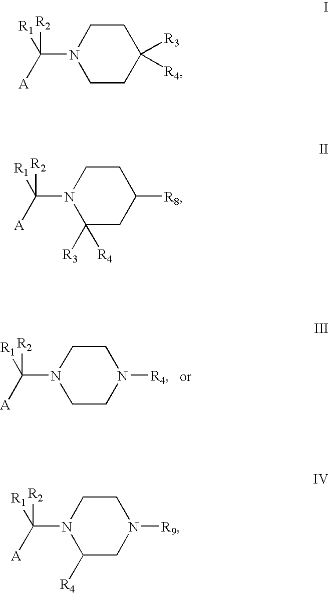
wherein:
R₁ and R₂ are each H or taken together are ═O;
R₃ is selected from OH, halogen, acyl, or substituted or unsubstituted C₁₋₆alkyl;
R₄ is selected from substituted or unsubstituted phenyl, substituted or unsubstituted thiophene, or ring B, wherein B is 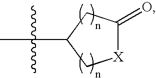
and ring B comprises 5, 6, or 7 atoms in the ring;

X is selected from O or NR₁₀;

R₁₀ is selected from H or substituted or unsubstituted C₁₋₆alkyl;

n is independently for each occurrence selected from 0, 1, 2, 3, or 4;

R₈ is selected from H, OH or substituted or unsubstituted C₁₋₆alkyl;

R₉ is selected from H or substituted or unsubstituted C₁₋₆alkyl;

A is selected from 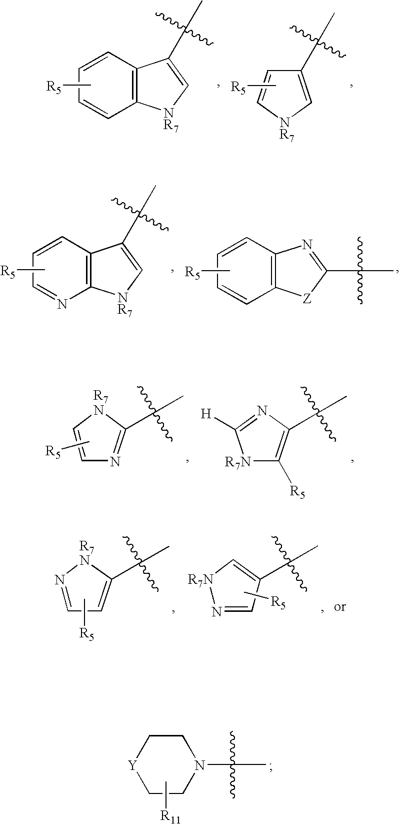
R₅ is independently for each occurrence selected from H, substituted or unsubstituted alkyl, alkenyl, alkynyl, aryl, aralkyl, alkoxy, aryloxy, aralkoxy, cycloalkyl, cycloalkoxy, heterocyclyl, heterocyclyloxy, heterocyclylalkyl, heteroaryl, or heteroaralkyl, keto, hydroxy, substituted or unsubstituted alkylthio, amino, alkylamino, alkanoylamino, aroylamino, or aralkanoylamino, carboxy, substituted or unsubstituted carboxyalkyl, carboxamidoalkyl, thiocarboxy, thiocarboxyalkyl, or thiocarboxamidoalkyl, halo, cyano, nitro, formyl, acyl, or substituted or unsubstituted sulfonyl, or sulfonamido, and is optionally substituted with 1-3 R₆ groups;
R₁₁ is independently for each occurrence selected from H, substituted or unsubstituted alkyl, alkenyl, alkynyl, aryl, aralkyl, alkoxy, aryloxy, aralkoxy, cycloalkyl, cycloalkoxy, heterocyclyl, heterocyclyloxy, heterocyclylalkyl, heteroaryl, or heteroaralkyl, keto, hydroxy, substituted or unsubstituted alkylthio, amino, alkylamino, alkanoylamino, aroylamino, or aralkanoylamino, carboxy, substituted or unsubstituted carboxyalkyl, carboxamidoalkyl, thiocarboxy, thiocarboxyalkyl, or thiocarboxamidoalkyl, halo, cyano, nitro, formyl, acyl, or substituted or unsubstituted sulfonyl, or sulfonamido, and is optionally substituted with 1-3 R₆ groups;
R₆ is independently for each occurrence selected from substituted or unsubstituted alkyl, aryl, aralkyl, alkoxy, aryloxy, heterocyclyl, or heterocyclyloxy, keto, hydroxy, substituted or unsubstituted alkylthio, amino, alkanoylamino, or aroylamino, carboxy, substituted or unsubstituted carboxyalkyl, or carboxamidoalkyl, halo, cyano, nitro, formyl, or substituted or unsubstituted sulfonyl, or sulfonamido;
Y is CH₂, O, S or NR₁₀;
Z is selected from O or NR₇; and
R₇ is independently for each occurrence selected from H or substituted or unsubstituted C₁₋₆alkyl.

In certain embodiments wherein the compound is represented by formula III and R₁ and R₂ are each H, R₅ and R₇ are each H.

In certain embodiments wherein the compound is represented by formula III, R₁ and R₂ taken together are ═O.

In certain embodiments of the invention, R₄ is 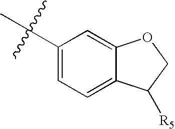
and R₅ is defined as above. In more specific embodiments, the compound is enriched in one of the stereoisomers at the site of attachment of R₅ to R₄.

In other embodiments of the invention, R₄ is 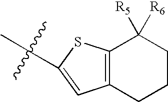
and R₅ and R₆ are defined as above. In more specific embodiments, the compound is enriched in one of the stereoisomers at the site of attachment of R₅ and R₆ to R₄.

In some embodiments of the invention, the compound is represented by structural formula II and is enriched in one of the stereoisomers at the site of attachment of R₃ and R₄.

In certain embodiments, R₃ is selected from OH, F, C(O)CF₃, or CH₃. In more specific embodiments, R₃ is OH.

In certain embodiments, R₈ is selected from H, OH or CH₃.

In certain embodiments, R₉ is selected from H or CH₃.

In some embodiments, the compound is 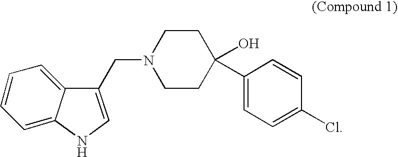
Piperidinomethyl-indole derivatives and the ability of these derivatives to antagonize dopamine activity have been described in U.S. Pat. No. 4,358,456. The utility of these derivatives in the treatment of psychoses, including schizophrenia, in a mammal have been proposed, but effects of the derivatives on the MC4 receptor or the utility of the derivatives in the treatment of obesity, eating disorders, or sexual dysfunction have not been reported.

In other embodiments of the invention, the compound is

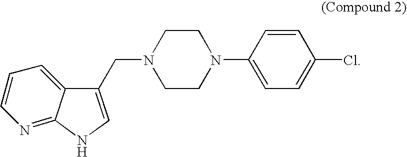

In some embodiments, the compound is neither of the two above-listed compounds.

The compounds of the invention (e.g., a compound of any one of formulae I to IV or a pharmaceutically acceptable salt thereof, or a solvate or prodrug of the compound or its salt) may be synthesized using conventional techniques as would be understood by the skilled artisan. Advantageously, these compounds are conveniently synthesized from readily available starting materials.

Certain compounds of the present invention (e.g., a compound of any one of formulae I to IV or a pharmaceutically acceptable salt thereof, or a solvate or prodrug of the compound or its salt) may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Methods of preparing substantially isomerically pure compounds are known in the art. If, for instance, a particular enantiomer of a compound of the present invention (e.g., a compound of any one of formulae I to IV or a pharmaceutically acceptable salt thereof, or a solvate or prodrug of the compound or its salt) is desired, it may be prepared by synthesis from optically pure precursors, asymmetric synthesis, or by derivation with a chiral auxiliary, where the resulting diastereomeric mixture is separated and the auxiliary group cleaved to provide the pure desired enantiomers. Racemates may also be resolved by chromatography, using, for example a chiral HPLC column. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers. Alternatively, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Techniques for inverting or leaving unchanged a particular stereocenter, and those for resolving mixtures of stereoisomers are well known in the art, and it is well within the ability of one of skill in the art to choose an appropriate method for a particular situation. See, generally, Furniss et al. (eds.), Vogel's Encyclopedia of Practical Organic Chemistry 5^{th} Ed., Longman Scientific and Technical Ltd., Essex, 1991, pp. 809-816; and Heller, Acc. Chem. Res. 23: 128 (1990).

As used herein, the compounds of the invention are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention, which, upon administration to a recipient, is capable of providing or provides (directly or indirectly) a compound of this invention.

Accordingly, this invention also provides prodrugs of the compounds of the invention, which are derivatives that are designed to enhance biological properties such as oral absorption, clearance, metabolism or compartmental distribution. Such derivations are well known in the art.

As the skilled practitioner realizes, the compounds of the invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism or alter rate of excretion.

Certain derivatives and prodrugs are those that increase the bioavailability of the compounds of the invention when such compounds are administered to an individual (e.g., by allowing an orally administered compound to be more readily absorbed into the blood), have more favorable clearance rates or metabolic profiles, or enhance delivery of the parent compound to a biological compartment (e.g., the brain or lymphatic system) relative to the parent species. Examples of prodrugs include derivatives in which a group that enhances aqueous solubility or active transport through the gut membrane is appended to the structure.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, trifluoroacetic, propionic, isobutyric, maleic, malonic, benzoic, succinic, suberic, fumaric, lactic, mandelic, phthalic, benzensulfonic, p-tolylsulfonic, citric, tartaric, methanesulfonic, and the like. Also included are the salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention may contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs form the various salt forms in certain physical properties, such as solubility in polar solvents, but otherwise the salts are equivalent to the parent form of the compound for the purposes of the present invention.

The pharmaceutically acceptable addition salts of the compounds of the invention may also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

The compounds of the invention may be administered as a pharmaceutical composition containing, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a specific embodiment, the pharmaceutical compositions have a low pyrogen activity to be suitable for use in a human patient. The excipients may be chosen, for example, to effect delayed release of an agent or to selectively target one or more cells, tissues or organs. The pharmaceutical composition may be in dosage unit form such as tablet, capsule, sprinkle capsule, granule, powder, syrup, suppository, injection or the like. The composition may also be present in a transdermal delivery system, e.g., a skin patch.

The term “low pyrogen activity”, with reference to a pharmaceutical preparation, refers to a preparation that does not contain a pyrogen in an amount that would lead to an adverse effect (e.g., irritation, fever, inflammation, diarrhea, respiratory distress, endotoxic shock, etc.) in a subject to which the preparation has been administered. For example, the term is meant to encompass preparations that are free of, or substantially free of, an endotoxin such as, for example, a lipopolysaccharide (LPS).

A pharmaceutically acceptable carrier may contain physiologically acceptable agents that act, for example, to stabilize or to increase the absorption of a compound of the instant invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition also may comprise a liposome or other polymer matrix, which may have incorporated therein, for example, a compound of the invention. Liposomes, for example, which consist of phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from one organ, or portion of the body, to another organ, or portion of the body. Each carrier must be “acceptable” in the sense of being compatible with the other ingredients of the formulation and not injurious to the patient. Some examples of materials that can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) water with a low pyrogen activity; (17) isotonic saline; (18) Ringer's solution; (19) ethyl alcohol; (20) phosphate buffer solutions; and (21) other non-toxic compatible substances employed in pharmaceutical formulations. See Remington: The Science and Practice of Pharmacy, 20th ed. (Alfonso R. Gennaro ed.), 2000.

A pharmaceutical composition containing a compound of the instant invention may be administered to a host by any of a number of routes of administration including, for example, orally (for example, drenches as in aqueous or non-aqueous solutions or suspensions, tablets, boluses, powders, granules, pastes for application to the tongue); sublingually; anally, rectally or vaginally (for example, as a pessary, cream or foam); parenterally (including intramusclularly, intravenously, subcutaneously or intrathecally as, for example, a sterile solution or suspension); nasally; intraperitoneally; subcutaneously; transdermally (for example as a patch applied to the skin); or topically (for example, as a cream, ointment or spray applied to the skin). The compound may also be formulated for inhalation. In certain embodiments, a compound of the instant invention may be simply dissolved or suspended in sterile water. Details of appropriate routes of administration and compositions suitable for same can be found in, for example, U.S. Pat. Nos. 6,110,973; 5,763,493; 5,731,000; 5,541,231; 5,427,798; 5,358,970; and 4,172,896, as well as in patents cited therein.

The formulations of the present invention may conveniently be presented in unit dosage form and may be prepared by any methods well known in the art of pharmacy. The amount of active ingredient that can be combined with a carrier material to produce a single dosage form will vary depending upon the host being treated and the particular mode of administration. The amount of active ingredient that can be combined with a carrier material to produce a single dosage form will generally be that amount of the compound that produces a therapeutic effect. Generally, out of one hundred percent, this amount will range from about 1 percent to about 99 percent of active ingredient, in some embodiments from about 5 percent to about 70 percent, and in more specific embodiments from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that may be used include polymeric substances and waxes. The active ingredient may also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions may also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal, vaginal, or urethral administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions may be formulated for delivery via a catheter, stent, wire, or other intraluminal device. Delivery via such devices may be especially useful for delivery to the bladder, urethra, ureter, rectum, or intestine.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a compound of this invention, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms may be made by dissolving or dispersing the compound in the proper medium. Absorption enhancers may also be used to increase the flux of the compound across the skin. The rate of such flux may be controlled by either providing a rate controlling membrane or dispersing the compound in a polymer matrix or gel.

Ophthalmic formulations, eye ointments, powders, solutions and the like, are also contemplated as being within the scope of this invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, chelators and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, may be used to form an implant for the sustained release of a compound at a particular target site.

It is contemplated that a compound of the present invention will be administered to a host (e.g., a mammal and, in specific embodiments, a human) in a therapeutically effective amount. By “therapeutically effective amount” is meant the concentration of a compound that is sufficient to elicit the desired therapeutic effect (e.g., treatment of obesity, treatment of an eating disorder, treatment of sexual dysfunction, etc.). It is generally understood that the effective amount of the compound will vary according to the weight, gender, age, and medical history of the host. Other factors that influence the effective amount may include, but are not limited to, the severity of the patient's condition, the disorder being treated, the stability of the compound, and, if desired, another type of therapeutic agent being administered with the compound of the invention. A larger total dose may be delivered by multiple administrations of the agent. Methods to determine efficacy and dosage are known to those skilled in the art. See, e.g., Roden, Harrison's Principles of Internal Medicine, Ch. 3, McGraw-Hill, 2004.

Actual dosage levels of the active ingredients in the pharmaceutical compositions of the invention may be varied so as to obtain an amount of the active ingredient that is effective to achieve the desired therapeutic response for a particular patient, composition, and mode of administration, without being toxic to the patient.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In specific embodiments, the active compound will be administered once daily.

It is contemplated that a therapeutically effective amount of the compound to be administered to a host (e.g., a mammal, in some embodiments, a human) in methods of the invention will be in the range of 1 mg/day and 100 mg/day. In certain embodiments, the therapeutically effective amount of the compound to be administered to a host in methods of the invention will be in a range of 1 mg/day and 60 mg/day. In more specific embodiments, the therapeutically effective amount of the compound to be administered to a host in methods of the invention will be in a range of 5 mg/day and 30 mg/day.

The host receiving this treatment is any mammal in need, including primates, and other mammals such as equines, cattle, swine and sheep. In certain embodiments, the host is a human. In certain other embodiments, the host is a mammalian pet.

In another embodiment of the invention, the compounds of the invention (e.g., a compound of any one of formulae I to IV or a pharmaceutically acceptable salt thereof, or a solvate or prodrug of the compound or its salt) are administered alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds may be administered either in the same formulation or in a separate formulation, either concomitantly or sequentially. Thus, an individual who receives such treatment may benefit from a combined effect of different therapeutic compounds.

In another aspect, the present invention provides a method of treating obesity, eating disorders, and sexual dysfunction, or a method of potentiating the effect of an MC4 receptor agonist in a patient in need of anti-psychotic treatment, comprising administering to said patient a compound of the invention (e.g., a compound of any one of formulae I to IV or a pharmaceutically acceptable salt thereof, or a solvate or prodrug of the compound or its salt). In another aspect, the present invention provides a method of treating obesity, eating disorders, and sexual dysfunction, or a method of potentiating the effect of an MC4 receptor agonist in a patient being treated with one or more anti-psychotic agents, comprising administering to said patient a compound of the invention (e.g., a compound of any one of formulae I to IV or a pharmaceutically acceptable salt thereof, or a solvate or prodrug of the compound or its salt). In certain embodiments of methods of the invention wherein a compound of the invention (e.g., a compound of any one of formulae I to IV or a pharmaceutically acceptable salt thereof, or a solvate or prodrug of the compound or its salt) is administered to a patient being treated with one or more anti-psychotic agents, the anti-psychotic agents are selected from any suitable anti-psychotic agent. Suitable anti-psychotic agents include, but are not limited to, clozapine, olanzapine, quetiapine, risperidone, ziprasidone, aripiprazole, trifluoperazine, flupenthixol, loxapine, perphenazine, chlorpromazine, haloperidol, fluphenazine decanoate, thioridazine, or a pharmaceutically acceptable salt thereof. In more specific embodiments, the anti-psychotic medication is an atypical anti-psychotic medication.

In certain other embodiments of methods of the invention, a compound of the present invention (e.g., a compound of any one of formulae I to IV or a pharmaceutically acceptable salt thereof, or a solvate or prodrug of the compound or its salt) is administered conjointly with an antagonist of the CB1 receptor. In more specific embodiments, the antagonist of the CBI receptor is norfluoxetine enriched for the (R) enantiomer.

In certain embodiments, the present invention relates to methods of treatment with norfluoxetine. In certain embodiments, the therapeutic preparation may be enriched to provide predominantly one enantiomer of norfluoxetine. An enantiomerically enriched mixture may comprise, for example, at least 60 mol percent of one enantiomer, or more preferably at least 75, 90, 95, or even 99 mol percent. In certain embodiments, norfluoxetine is enriched in the (R) enantiomer. In certain embodiments, (R)-norfluoxetine is substantially free of the (S)-enantiomer, wherein substantially free means that the substance in question makes up less than 10%, or less than 5%, or less than 4%, or less than 3%, or less than 2%, or less than 1% as compared to the amount of the (R)-enantiomer, e.g., in the composition or compound mixture. For example, if a composition or compound mixture contains 98 grams of the (R)-enantiomer and 2 grams of the (S)-enantiomer, it would be said to contain 98 mol percent of the (R)-enantiomer and only 2% of the (S)-enantiomer. In certain embodiments, norfluoxetine is provided as a salt of norfluoxetine or a solvate of norfluoxetine or its salt.

In still other embodiments, a compound of the present invention is administered conjointly with an agonist of the MC4 receptor.

In certain embodiments of methods of the invention, a compound of the present invention (e.g., a compound of any one of formulae I to IV or a pharmaceutically acceptable salt thereof, or a solvate or prodrug of the compound or its salt) may be administered conjointly with an inhibitor of dopamine reuptake. In certain other embodiments, a compound of the present invention (e.g., a compound of any one of formulae I to IV or a pharmaceutically acceptable salt thereof, or a solvate or prodrug of the compound or its salt) may be administered conjointly with an inhibitor of norepinephrine reuptake. In still other embodiments, a compound of the present invention (e.g., a compound of any one of formulae I to IV or a pharmaceutically acceptable salt thereof, or a solvate or prodrug of the compound or its salt) may be administered conjointly with an inhibitor of both dopamine and norepinephrine reuptake.

In another embodiment of methods of the invention, a compound of the present invention (e.g., a compound of any one of formulae I to IV or a pharmaceutically acceptable salt thereof, or a solvate or prodrug of the compound or its salt) may be administered conjointly with a dopamine agonist or partial agonist.

Specific compounds that may be conjointly administered with a compound of the present invention (e.g., a compound of any one of formulae I to IV or a pharmaceutically acceptable salt thereof, or a solvate or prodrug of the compound or its salt) include, but are not limited to, bupropion, methylphenidate, sibutramine, sertraline, venlafaxine, atomoxetine, amineptine, benztropine or reboxetine or metabolites or stereoisomers thereof.

Methods of Potentiation of an MC4 Receptor Agonist

In another aspect, the instant invention relates to methods of potentiating the effect of an MC4 receptor agonist in a mammalian host. In certain embodiments, the methods of the present invention relate to allosterically potentiating the effect of an MC4 receptor agonist in a mammalian host. Accordingly, these methods result in an increased effective activity of the MC4 receptor agonist in the mammalian host. In certain specific embodiments, the potentiating compound itself lacks direct agonist activity against the MC4 receptor. In one embodiment, the methods of the invention comprise administering to the mammalian host a compound that lowers the EC₅₀ of the agonist for the MC4 receptor. In some embodiments, the compound lowers the EC₅₀ by at least 10%. In specific embodiments, the compound lowers the EC₅₀ by at least 20%, at least 30%, or at least 40%. In more specific embodiments, the compound lowers the EC₅₀ by at least 50% or at least 60%. In even more specific embodiments, the compound lowers the EC₅₀ by at least 70%, at least 80%, or even more.

In another embodiment, the methods of the invention comprise administering to the mammalian host a compound that increases the maximum effect of the agonist on the MC4 receptor. In some embodiments, the compound increases the maximum effect by at least 10%. In certain embodiments, the compound increases the maximum effect by at least 20%, at least 30%, or at least 40%. In more specific embodiments, the compound increases the maximum effect by at least 50% or at least 60%. In even more specific embodiments, the compound increases the maximum effect by at least 70%, at least 80%, or even more.

The EC₅₀ of an agonist for an MC4 receptor, the maximum effect of an agonist on an MC4 receptor, and the effect of a subject potentiator compound on these values may be readily determined by one of skill in the art, for example as described in the example that follows. Other methods for measuring the ability of a compound to potentiate the effect of an agonist on an MC4 receptor will likewise be apparent to the skilled artisan. It is thus within the skill in the art to identify and use novel compounds capable of potentiating the effect of an MC4 receptor agonist according to the methods of the instant invention.

In certain specific embodiments, the MC4 receptor agonist is α-MSH or NDP α-MSH.

In some embodiments, the invention relates to methods of potentiating the effect of an MC4 receptor agonist in a mammalian host, wherein the host suffers from obesity, an eating disorder, or sexual dysfunction, and wherein the potentiating compound is administered in an effective dose.

In specific embodiments, the methods of potentiating the effect of an MC4 receptor agonist in a mammalian host comprise administering a compound of the invention (e.g., a compound of any one of formulae I to IV or a pharmaceutically acceptable salt thereof, or a solvate or prodrug of the compound or its salt).

In some embodiments, the compound that potentiates the effect of an MC4 receptor agonist in a mammalian host also attentuates the effect of an MC4 receptor inverse agonist. In some embodiments, the compound is an allosteric potentiator of the MC4 receptor agonist. In certain such embodiments, the compound is an allosteric attenuator of an MC4 receptor inverse agonist.

Methods of Attenuation of an MC4 Receptor Inverse Agonist

In another aspect, the instant invention relates to methods of allosterically attenuating the effect of an MC4 receptor inverse agonist in a mammalian host.

Accordingly, these methods result in a decreased effective activity of the MC4 receptor inverse agonist in the mammalian host. In certain specific embodiments, the attenuating compound itself lacks direct inverse agonist activity against the MC4 receptor. In one embodiment, the methods of the invention comprise administering to the mammalian host a compound that raises the EC₅₀ of the inverse agonist for the MC4 receptor. In some embodiments, the compound raises the EC₅₀ by at least 10%. In specific embodiments, the compound raises the EC₅₀ by at least 20%, at least 30%, or at least 40%. In more specific embodiments, the compound raises the EC₅₀ by at least 50% or at least 60%. In even more specific embodiments, the compound raises the EC₅₀ by at least 70%, at least 80%, or even more.

In another embodiment, the methods of the invention comprise administering to the mammalian host a compound that decreases the maximum effect of an inverse agonist on the MC4 receptor. In some embodiments, the compound decreases the maximum effect by at least 10%. In certain embodiments, the compound decreases the maximum effect by at least 20%, at least 30%, or at least 40%. In more specific embodiments, the compound decreases the maximum effect by at least 50% or at least 60%. In even more specific embodiments, the compound decreases the maximum effect by at least 70%, at least 80%, or even more.

The EC₅₀ of an inverse agonist for an MC4 receptor, the maximum effect of an inverse agonist on an MC4 receptor, and the effect of a subject allosteric attenuator compound on these values may be readily determined by one of skill in the art. It is thus within the skill in the art to identify and use novel compounds capable of allosterically attenuating the effect of an MC4 receptor inverse agonist according to the methods of the instant invention.

In certain specific embodiments, the MC4 receptor inverse agonist is agouti-related peptide (AgRP).

In specific embodiments, the methods of allosterically attenuating the effect of an MC4 receptor inverse agonist in a mammalian host comprise administering a compound of the invention (e.g., a compound of any one of formulae I to IV or a pharmaceutically acceptable salt thereof, or a solvate or prodrug of the compound or its salt).

It will be readily apparent to one of ordinary skill in the relevant arts that other suitable modifications and adaptations to the methods and applications described herein may be made without departing from the scope of the invention or any embodiment thereof. Having now described the present invention in detail, the same will be more clearly understood by reference to the following Example, which is included herewith for purposes of illustration only and is not intended to be limiting of the invention.

### EXAMPLES

### Example 1

### Identification and Characterization of MC4 Potentiators Assays

The cDNA for the human Melonocortin-4 (MC4) receptor was sub-cloned into the pBS397 vector and then transfected into HEK293 cells obtained from ATCC. Transfected cells were carried under selection with the antibiotic G418 (neomycin) for three weeks.

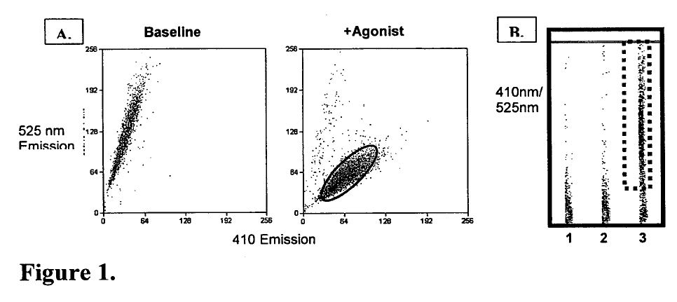

Cell line optimization was done using an automated Direct Sample Injection System (“DSIS”) in conjunction with Fluorescence Activated Cell Sorting (“FACS”). The DSIS is described in more detail in U.S. Patent Application No. 2005/0249635, which is incorporated herein by reference in its entirety. Cells were sorted according to a functional response. Intracellular Ca²⁺ (Ca²⁺ᵢ) levels were measured with Ca²⁺ chelating fluorescent probes. Here, the single excitation (UV excitation source), dual emission probe, Indo-1 (Invitrogen/Molecular Probes) was used. The Indo-1 emissions (410 nm/525 nm) increased and decreased, respectively, as the Ca²⁺ᵢ levels rose. The ratio of 410 nm to 525 nm emissions thus provided a stable index of Ca²⁺ᵢ that was fundamentally independent of the extent of dye loading. FIG. 1A shows dot plots of cells loaded with Indo-1, displaying low Ca²⁺ᵢ emissions at baseline compared to high Ca²⁺ᵢ emission seen with the addition of agonist. The oval represents the type of gate that can be set as a sorting criterion.

MC4 cells were plated 24-48 hours in advance, and were harvested with trypsin at ˜80% confluency. Cells were then centrifuged and resuspended two times in Hybridoma Media, the final time at a concentration of 1×10⁶ cells/mL. Two μM of Indo-1 was added and cells were incubated for 1 hour on a rotator at room temperature. Cells were washed two times and resuspended at a concentration of 2×10⁶ cells/mL in Hybridoma Media. The MC4 agonist, α-Melanocyte Stimulating Hormone (α-MSH, Sigma), was prepared at a concentration which was 4 times the Emax concentration for the MC4 cells. Twenty μL of α-MSH was placed in 8 rows of a 384-well plate. The probe-loaded cells were then placed in the cell suspension system on the DSIS where they were continuously rocked to keep them in a suspended state. The α-MSH plate was also transferred to the DSIS-FACS.

The intrinsic heterogeneity of individual cells within a cell population of a single cell line was exploited in searching for the desired functional assay response, by imposing it as the selection criterion. This cellular evolution approach was used to select the subset of cells that couple the transfected GPCR protein to a desired functional assay readout. Here, the MC4 cells that responded to α-MSH with an increase in Ca²⁺ᵢ were sorted. FIG. 1B shows a dot plate displaying cells as run through DSIS with the Indo-1 emissions displayed as a ratio (y-axis) over time x-axis). Samples 1 and 2 were at baseline levels while 3 displayed the response seen in the presence of agonist (dashed rectangle).

To run the sorting assay, DSIS added 60 μL of cells to one well of α-MSH in the 384-well plate. This was done at an injection rate of 40 μL/second, which mixed the cells with the compound. The sample was then injected into a MoFlo cytometer (Dako-Cytomation). Using Summit software, dot plots that display the ratio of the 410 nm and 525 nm emissions of the Indo-1 probe were used to set a gate for cells displaying a high Ca²⁺ᵢ response. Cells were injected into the cytometer for 45 seconds each round. This process continued iteratively until all cells were sorted. Cells passing the sort criteria were deflected into a 5 mL collection tube containing 2 mL of FBS. Once the sort was complete, the cells were transferred into a new tissue culture flask and the sorted population was expanded. The new, sorted MC4 population was then prepared for testing, loaded with Indo-1 and analyzed for Ca²⁺ᵢ response. The complete sorting procedure was repeated until a cell line was developed that had a response rate greater than 70%. For the MC4 cell line, the initial response rate was 32% and after 3 sorts increased to 72% of the cells. In addition to collecting a population of sorted cells, the Cyclone adaptor to the MoFlo cytometer sorted a single cell into each well of a 96-well plate for clonal sorting. The resulting individual clonal populations were then assayed for Ca²⁺ᵢ response. One clonal MC4 population with a Ca²⁺ᵢ response rate of 75% was chosen for subsequent screening assays.

MC4 Allosteric Modulator Screening

The screening process assayed both the clonal MC4 (cMC4) and the Control cell line (HEK293 cells mock-transfected with the pBS397 vector minus the MC4 insert) simultaneously. This was done by staining one population with a tracker dye. The system used herein consists of an initial treatment with Biotin-X DHPE (Invitrogen/Molecular Probes), a phospholipid conjugated to biotin. The phospholipid portion inserted into the cell membrane leaving the biotin exposed on the cell surface. This was followed by a secondary treatment with an Alexa dye conjugated to streptavidin. The populations were then distinguished by their respective fluorescent signatures.

The cMC4 and Control Cells were plated 24-48 hours in advance, and were harvested with trypsin at ˜80% confluency. Cells were then centrifuged and resuspended two times in Hybridoma Media, the final time at a concentration of 1×10⁶ cells/mL. Both cell lines were then loaded with 2 mM of Indo-1 plus 3 μg/mL Biotin-X DHPE and then were incubated for 1 hour on a rotator at room temperature. Cells were washed two times and resuspended at a concentration of 1×10⁶ cells/mL in Hybridoma Media. The cMC4 cell line then got 2 μg/ml of Alexa 488-streptavidin (Invitrogen/Molecular Probes). Cells were incubated for an additional 30 minutes on a rocker at room temperature. Both cell lines were centrifuged and washed 2 times in Hybridoma Media with the final resuspension at 5×10⁵ cells/mL.

A library of compounds for screening was set up in 96-well V-bottom plates. Each plate held 80 compounds located in columns 2-11. Compounds were initially solubilized in DMSO, and then were diluted with PBS. The final assay plates had 20 μL/well of 50 μM compound (in PBS+1% DMSO). Columns 1 and 12 contained PBS+1% DMSO and were used as Background and Control wells.

The probe-loaded cMC4 and Control cell mixture was placed in the Cell Suspension System on the DSIS where they were continuously rocked to keep them in a suspended state. To screen for allostenic modulators, an EC₅₀ concentration of a natural ligand of the receptor was used as a control response. Each day a new aliquot of α-MSH was used to prepare a dose/response determination plate. Ten α-MSH concentrations were used starting at 30 μM, then diluted at half log intervals down to 1 nM. Each well contained 20 μL of α-MSH to which 60 μL of cells were added. As the α-MSH in the plate was diluted by the cell suspension, the concentration in the plate was four times what was required to achieve the final concentration for each dose. For this initial determination, DSIS was set to agonist mode. Screening assays were run on the CyAn Cytometer (Dako-Cytomation). DSIS added 80 μL of cells to the first well of the dose/response plate, the mixture incubated for 13 seconds, then was injected into the CyAn. This was repeated for each well. The DSIS software, NVS Sampler, recorded a timing file and the CyAn software, Summit, recorded a data file. These two files were then compiled and analyzed by NVS Analyzer (see below) to determine the percent of cells that responded to each concentration. The data were transferred to GraphPAD Prism (GraphPad Software, San Diego, Calif.) for a non-linear regression curve fit that determined the EC₅₀. To set up the allosteric screen, 5 mL of α-MSH was prepared, from the same aliquot used for the dose/response assay, at a concentration 5× the EC₅₀.

The allosteric screening assay was conducted with DSIS set in antagonist mode with preincubation. The cells were in place, the α-MSH (5×EC₅₀) was added to the appropriate vial holder and the first compound plate was in place. Each plate was run in two segments, rows 1-4 then rows 5-8. Each plate and segment had an individual code that was entered at the start of each run. The parameters of this screen included a 2 minute incubation after 60 μL of cells were added to the compound well. The α-MSH, 20 μL, was then added to the well and there was another 13-second incubation. The cell mixture was then injected into the CyAn for a 45 second interrogation. Wells in column one had no compound and did not receive agonist. This was our background or baseline measurement. Wells in column 12 had no compound, but got the EC₅₀ concentration of α-MSH. These were the control wells that were used to determine if a compound had a potentiating or attenuating effect. Subsequent plates were screened accordingly.

Analysis

When the screen was complete, the data files and the timing files were analyzed using NVS Analyzer. This single-cell data analysis software was designed to separate data coming from the flow cytometer into groups of cells and assign well numbers of compounds that were mixed with cells in each group. To accomplish this, the DSIS control software, NVS Sampler, recorded the time of each injection event. When the data were processed, the time of first injection was used to offset all injection events so that the first event occurred at time zero. The data from the NVS Sampler file along with the data from CyAn file were then loaded into NVS Analyzer. Cells with a timestamp between the first injection time and second injection time were assigned to the first processed well, then cells between second and third injections were assigned to the next processed well and so on. The second step of the analysis processes was to calculate the mean and standard deviation of intensity of a control well, with no compound added. This was done for each population separately since different population often have different background values due to variance in cell loading. A threshold was then set as the mean plus a user specified number of standard deviations. All cells with a response above this threshold were counted as activated. The number of cells activated for a particular population divided by the number of cells in that population was the percent responding value that could be plotted on a chart or exported into an ASCII file for import into ActivityBase.

Once the data was compiled, it was exported into the ActivityBase database system and the SARgen query tool was used for final analysis, hit detection and formatting. An average response for the control wells in each plate segment was determined, and the compound wells in that segment were compared to that average. A compound that elicited a response that was more than 25% plus or minus the average of the control, was determined a hit. Any compound that affected the control cell line was deleted from the list.

Validation

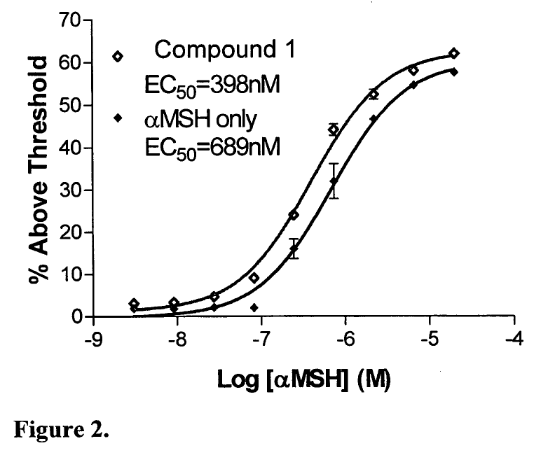

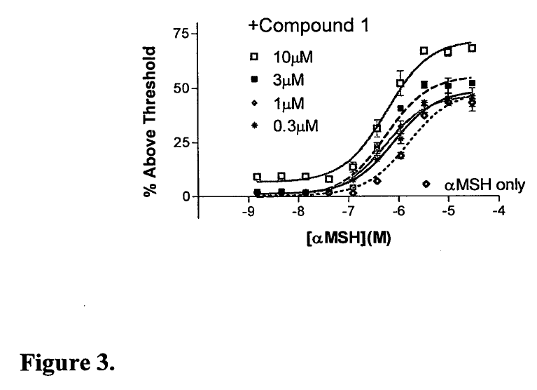

Compounds determined as hits from the primary screen were cherry picked into new plates for a secondary screen. This was done using DSIS in agonist mode as outlined above in the EC₅₀ determination. Control and cMC4 cells were prepared using the same protocol as used for the primary screening assay. DSIS added the cell suspension to the wells then transferred the mixture directly to the CyAn for analysis. This was done to determine if an observed effect was due to direct agonist activity of the compound. As an allosteric potentiator for the MC4 receptor was preferentially sought, the hit list was ideally comprised of those compounds that displayed a potentiation effect in the primary screen and lacked an agonist response in the secondary screen. These compounds were then used in dose-response experiments. α-MSH dose-response determination plates were prepared as outlined above and were run +/− the compounds of interest. FIG. 2 displays a left shift in the curve that would be expected of an allosteric potentiator. The compounds that displayed a left shift in the EC₅₀ of α-MSH at a concentration of 1-10 μM were run again in the presence of various concentrations of the compound (FIG. 3 and Table I). Two compounds advanced to this point, Compound 1 and Compound 2. They were then subjected to further validation experiments.

TABLE I
Effect of Compound 1 on the EC₅₀ of α-MSH

| [Compound 1] | EC50 |
|---|---|
| 0 μM | 1.566 × 10−6 |
| 0.3 μM | 7.949 × 10−7 |
| 1 μM | 5.291 × 10−7 |
| 3 μM | 5.394 × 10−7 |
| 10 μM | 5.738 × 10−7 |

Dissociation Kinetics

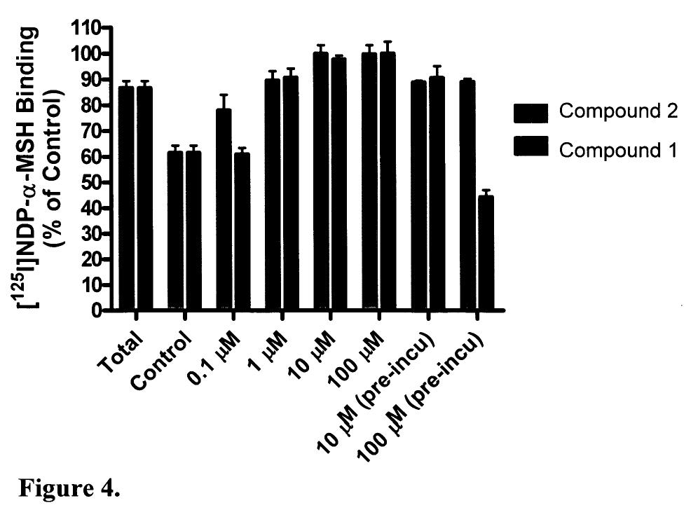

Dissociation kinetics assays were performed with the MC4 receptor agonist [¹²⁵I]-[Nle⁴, D-Phe⁷]-α-melanocyte stimulating hormone ([¹²⁵I]-NDP-α-MSH) (2 nM) in the binding buffer containing 33 mM Hepes, pH 7.5, 1 mM MgCl₂, 2.5 mM CaCl₂, 0.5% BSA and 0.25% bacitracin using HEK293 cell membranes stably expressing human MC4 receptors in 96-well plate format. The MC4 receptor membranes (10 μg/well) were incubated with 2 nM [¹²⁵I]-NDP-α-MSH in 100 μl binding buffer at room temperature for 2 h. Dissociation was initiated with addition of 100 μl unlabeled NDP-α-MSH (10 μM) in binding buffer in the absence or presence of different concentrations of compounds (Compound 1 or Compound 2). Dissociation was carried out at room temperature for indicated time. To determine the non-specific binding, experiments were also performed in the presence of 1 μM unlabeled NDP-α-MSH. Binding was terminated by addition of cold binding buffer and filtrated on Whatman GF/B glass-fiber filters using a sampling manifold. The filters were washed 6 times with cold binding buffer and air-dried overnight. The radioactivity was quantified on a TopCounter (PerkinElmer) after adding 1 μM unlabeled NDP-α-MSH (FIG. 4). Inhibition of the off-rate of the MC4 peptide ligand, ¹²⁵I-NDP-α-MSH, was diagnostic of an allosteric modulator.

Competition Binding Assays

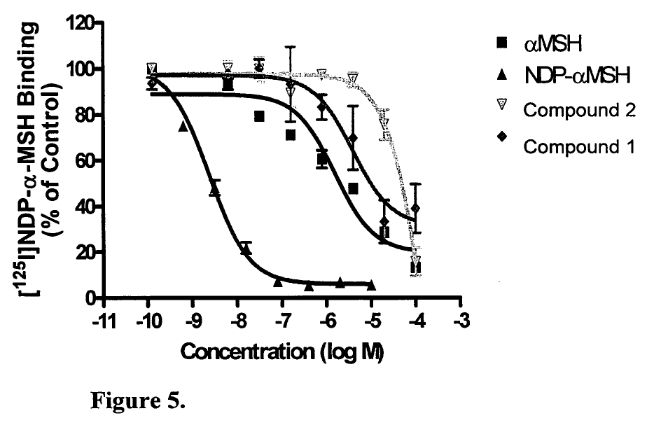

Competition ligand binding assays were performed with the MC4 receptor agonist [¹²⁵I]-[Nle⁴, D-Phe⁷]-α-melanocyte stimulating hormone ([¹²⁵I]-NDP-α-MSH) (2 nM) in the binding buffer containing 33 mM Hepes, pH 7.5, 1 mM MgCl₂, 2.5 mM CaCl₂, 0.5% BSA and 0.25% bacitracin using HEK293 cell membranes stably expressing human MC4 receptors in 96-well plate format. The MC4 receptor membranes (10 μg/well) were pre-incubated with different concentrations of tested compounds in the binding buffer at room temperature for 30 min prior to addition of 2 nM [¹²⁵I]-NDP-α-MSH in a final volume of 100 μl. The binding was carried out at room temperature for another 2 h. To determine the non-specific binding, experiments were also performed in the presence of 1 μM unlabeled NDP-α-MSH. Binding was terminated by addition of cold binding buffer and filtration on Whatman GF/B glass-fiber filters using a sampling manifold. The filters were washed 6 times with cold binding buffer and air-dried overnight. The radioactivity was quantified on a TopCounter after adding scintillation fluid. Specific binding was defined as the difference between the binding in the presence and absence of 1 μM unlabeled NDP-α-MSH. Data were analyzed by non-linear regression using program GraphPAD Prism (FIG. 5). Both NVS compounds partially competed binding of [³H] NDP-α-MSH to the MC4 receptor at high concentrations. Binding data obtained from these experiments are summarized in Table II.

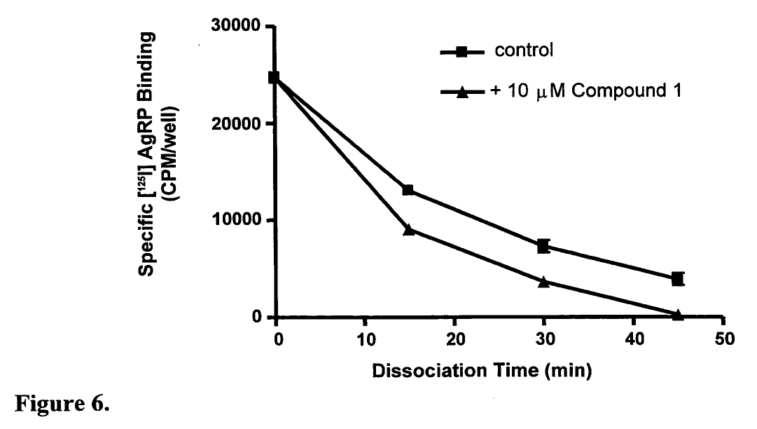

Similar experiments were performed with the MC4 receptor inverse agonist AgRP. FIG. 6 shows that compound I increases the dissociation of AgRP from the MC4 receptor.

TABLE II
Effect of specific compounds on the binding of [³H]
NDP-α-MSH to the MC4 receptor

| Compound | EC₅₀ (M) |
|---|---|
| αMSH | 1.564 × 10⁻⁶ |
| NDP-αMSH | 2.463 × 10⁻⁹ |
| Compound 2 | 0.0002516 |
| Compound 1 | 3.955 × 10⁻⁶ |

cAMP Assay

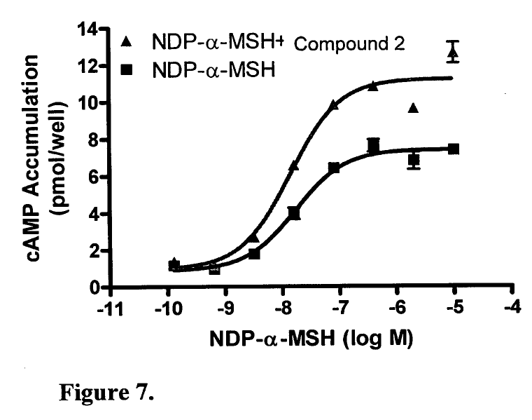

ELISA-based cAMP assay was used to determine ligand-induced cAMP accumulation in HEK293 cells stably expressing human MC4 receptors in 96-well plate format. Compounds and cAMP standards were diluted in DMEM (without FBS and phenol red) containing 1 mM IBMX. Cells were plated in T-75 flasks and cultured for 24 h before use. Cells were removed from tissue culture flasks using enzyme-free cell dissociation buffer, washed and resuspended in DMEM (without FBS and phenol red) containing 1 mM IBMX at a density of 1×10⁶ cells/ml. Cells (25,000/well) were added into 96-well white ELISA plate pre-coated with protein A, and incubated with tested compounds (Compound 1 and Compound 2) at room temperature for 30 prior to addition of agonists (α-MSH or NDP-α-MSH) in a volume of 50 μl. After incubation for 1 h, 50 μl of lysis buffer (0.5% NP-40 in TBS) was added, and incubated for 30 min. After adding rabbit anti-cAMP antibody (Sigma) diluted in TBST (TBS containing 0.05% Tween 20) (25 μl/well) and incubating for 30 min, 25 μl of cAMP-HRP conjugate dilution (in TBST) was added, and incubated for another 1 h. The plate was washed 6 times with TBST. To each well 200 μl of substrate (SuperSignal ELISA Pico Chemiluminesent substrate, Pierce, #37070) was added. The chemiluminescence activity was measured by a luminescence plate reader (NOVAstar, BMG Labtech). cAMP amount for each sample was estimated from cAMP standard curve. Data were analyzed by non-linear regression using program GraphPAD Prism (FIG. 7) and are summarized in Table III. This assay demonstrated the ability of Compound 2 to potentiate the response of NDP-α-MSH.

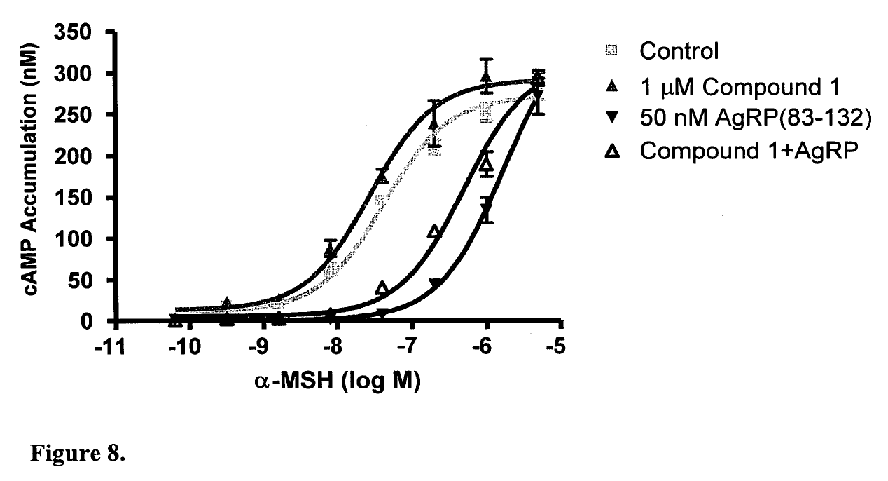

FIG. 8 shows that compound 1 potentiates α-MSH activity and attenuates AgRP activity.

TABLE III
Results of ELISA-based cAMP assay

| | NDP-α-MSH | NDP-α-MSH + Compound 2 |
|---|---|---|
| Bottom | 0.8564 | 0.9444 |
| Top | 7.415 | 11.23 |
| Log EC₅₀ (log M) | −7.770 | −7.858 |
| EC₅₀ (M) | 1.698 × 10⁻⁸ | 1.386 × 10⁻⁸ |

Single-Well Kinetics

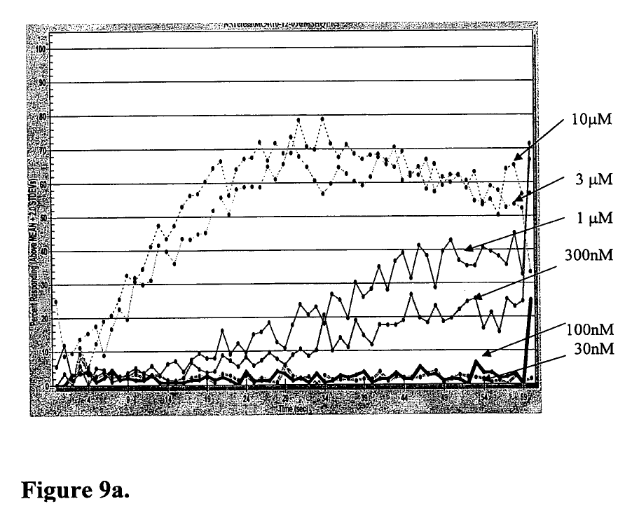

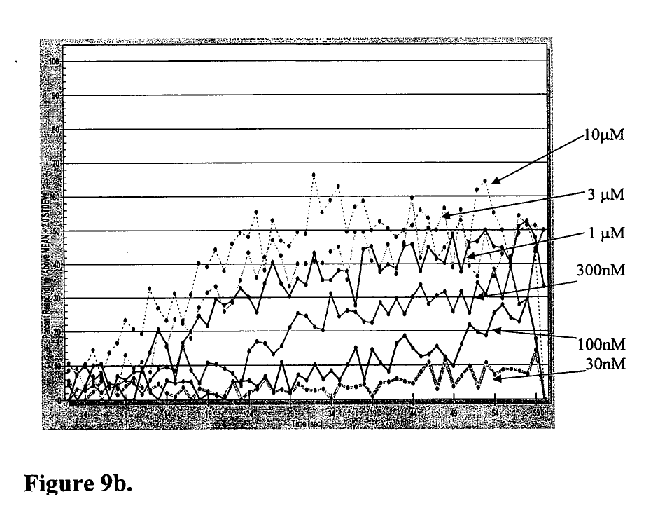

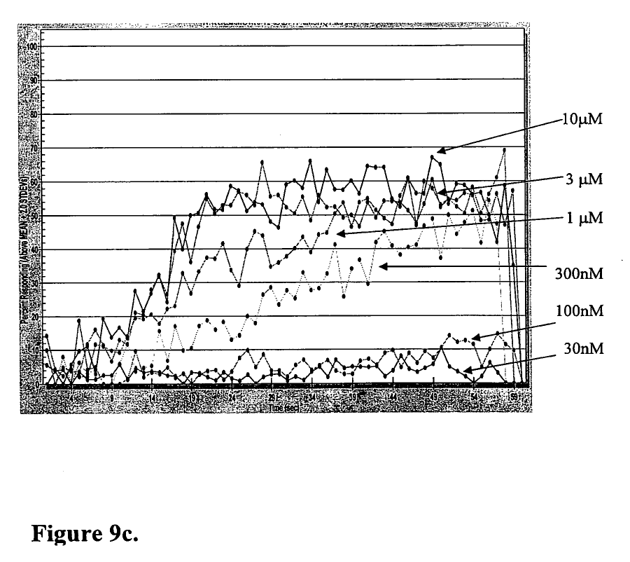

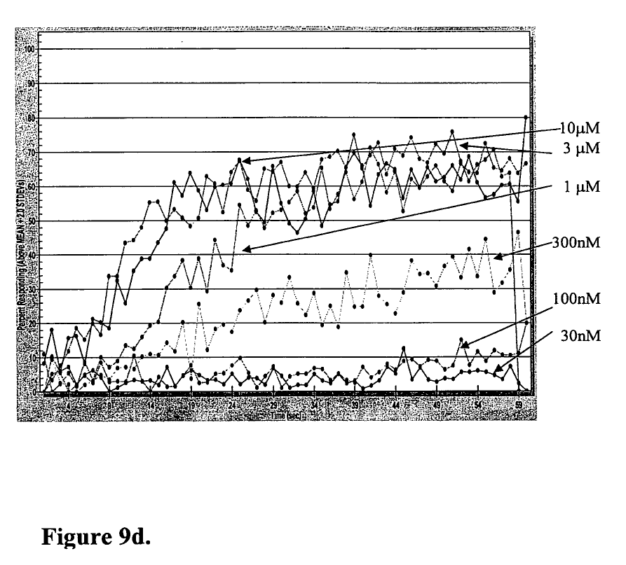

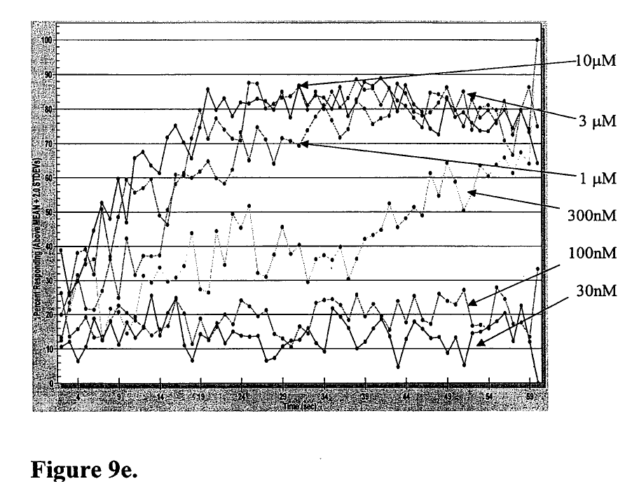

FIGS. 9A-9E show response kinetics from single-well assays. The response was initiated by αMSH with or without Compound 1. At low concentrations of Compound 1, the maximum peak response did not appreciably increase, but it was sustained at 3 μM and 10 μM αMSH. Whereas at 1 μM and 300 nM, αMSH onset and peak were increased and sustained. The onset and peak response increased with 3 μM and 10 μM concentrations of Compound 1 at all concentrations of αMSH. At 10 μM Compound 1 the activity of 100 nM and 30 nM concentrations of αMSH may be the result of low level activity induced by Compound 1 (FIG. 9E).

### Example 2

### Effect of Compound 1 on the Body Weight and Food Intake of Wild-Type and MC4 Knockout Male C57BL/6J Mice which Exhibit Obesity

The goal of this study was to investigate whether repeated administration of Compound 1 alters the body weight and daily food intake in wild-type and MC4 knockout C57BL/6J mice exhibiting obesity.

Mice were dosed at 40 mg/kg for two days with vehicle or test drug. Body weight and food and water intake were recorded daily.

Drugs:

The test compound was ground while being diluted in 1% methylcellulose, and the final solution was sonicated to give a uniform suspension suitable for dosing. Drug solutions were made up fresh each day 1-2 h before dosing and were administered using a dose volume in the range of 1-3 ml/kg.

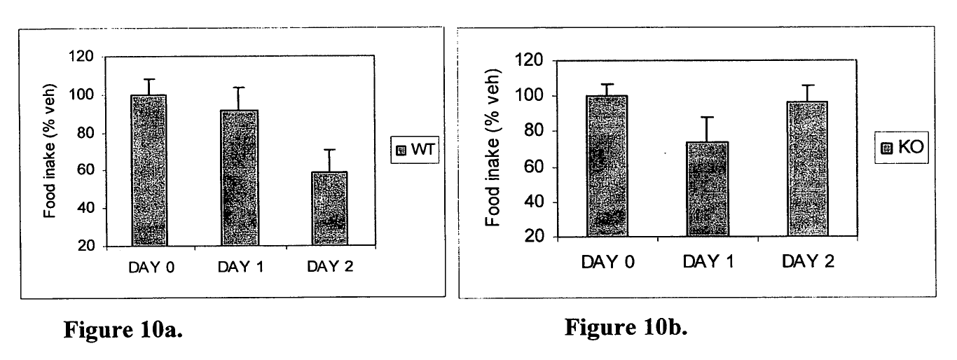

FIG. 10a shows the results of oral administration of 40 mg/kg of Compound 1 on food intake of diet-induced obese male wild-type C57BL/6J mice as a percent as compared to vehicle. FIG. 10b shows the results of oral administration of 40 mg/kg of Compound 1 on food intake of diet-induced obese male MC4 knockout C57BL/6J mice as a percent as compared to vehicle. Drug treatment commenced on Day 1. Treatment of wild-type mice showed approximately a 40% reduction in food intake as compared to vehicle on day 2. Treatment of MC4 knockout mice did not show a similar reduction.

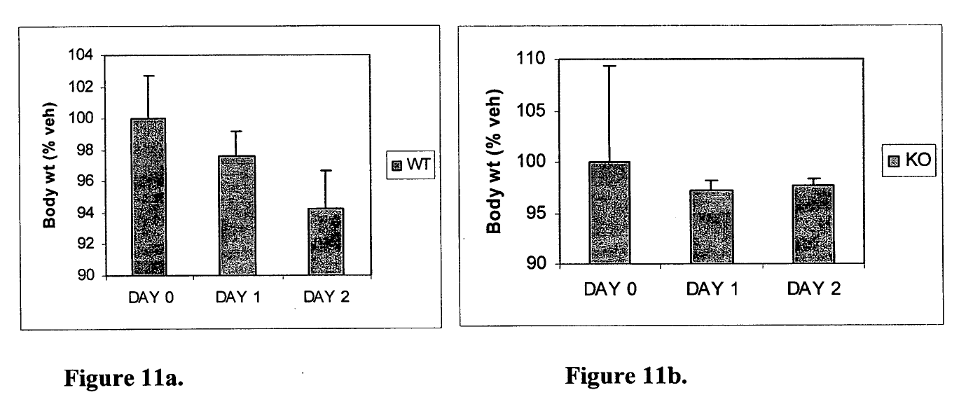

FIG. 11a shows the results of oral administration of 40 mg/kg of Compound 1 on body weight of diet-induced obese male wild type C57BL/6J mice as a percent as compared to vehicle. FIG. 11b shows the results of oral administration of 40 mg/kg of Compound 1 on body weight of diet-induced obese male MC4 knockout C57BL/6J mice as a percent as compared to vehicle. Drug treatment commenced on Day 1. Treatment of wild-type mice showed a statistically significant decrease of body weight as compared to vehicle on day 2. Treatment of MC4 knockout mice did not show a similar decrease.

### Example 3

### Effect of Treatment X on the Body Weight Food and Water Intake of Male C57BL/6J Mice Which Exhibit Diet Induced Obesity

The goal of this study was to investigate whether repeated administration of Treatment X, a 1:1 w:w combination of R-norfluoxetine hydrochloride and Compound 1, alters the body weight and daily food and water intake in C57BL/6J mice exhibiting obesity due to access to a high fat diet. Sibutramine, which is currently used clinically, and rimonabant, which has recently received regulatory approval for the management of obesity were used as reference compounds.

Animals:

C57BL/6J mice (7-8 weeks of age) were ordered from Charles River, Margate, Kent. Mice were group housed in polypropylene cages with free access to a high fat diet (D12451 45% of Kcal derived from fat; Research Diets, New Jersey, USA) and tap water at all times. Animals were maintained at 21±4° C. and 55±20% humidity on a normal phase 12 h light-dark cycle (lights on 04:30 h)

Experimental Procedures:

Animals were exposed to the high fat diet for 16 weeks. During this time body weight was recorded weekly. At the end of 14 weeks animals were singly housed in polypropylene cages for a further two week period (weeks 14-16) and placed on reverse phase lighting (lights off for 8 h from 9.30-17.30 h) during which time the room was illuminated by red light. Animals were dosed with vehicle orally throughout the baseline period. Body weight and food and water intake was recorded daily. Towards the end of the baseline period animals were allocated to groups. Upon completion of the baseline period, mice were dosed for 28 days with vehicle or test drug.

Body weight and food and water intake were recorded daily. Following drug administration the animals were examined and any overt behavior was recorded. For all dosing, the morning session was timed such that approximately half the mice were dosed at the time of lights out (09:30).

Drugs:

The test compounds were dissolved in 1% methylcellulose. Drug solutions were made up fresh each day 1-2 h before dosing and were administered using a dose volume in the range of 1-3 ml/kg. Drug doses were expressed as free base.

Data and Statistical Analysis:

Resulting body weights, food intake and water intake were expressed as mean values±SEM, and the SEMs are calculated from residuals of the statistical model. Body weight data was analysed by ANCOVA with Day 1 as covariate followed by appropriate comparisons (two-tailed) to determine significant differences from the control group. P<0.05 was considered to be statistically significant. Daily food and water intake data was analysed by ANOVA.

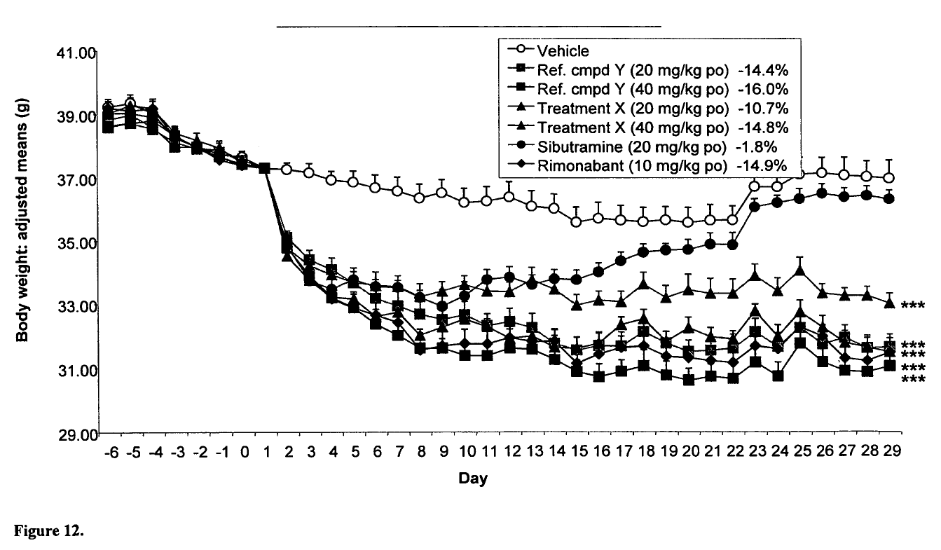

FIG. 12 shows the results of oral administration of Treatment X, Sibutramine and Rimonabant on the body weight of diet-induced obese male C57BL/6J mice. Drug treatment commenced on Day 1. Treatment X dosed at 20 mg/kg and 40 mg/kg and Rimonabant dosed at 10 mg/kg all demonstrated statistically significant weight reduction as compared to vehicle on day 29. Oral administration of Treatment X at 20 mg/kg and 40 mg/kg resulted in a 10 and 14% reduction of body weight respectively. This compares to only a 2% reduction in body weight for sibutramine administered at 20 mg/kg, and is comparable to the 15% reduction in body weight for rimonabant administered at 10 mg/kg.

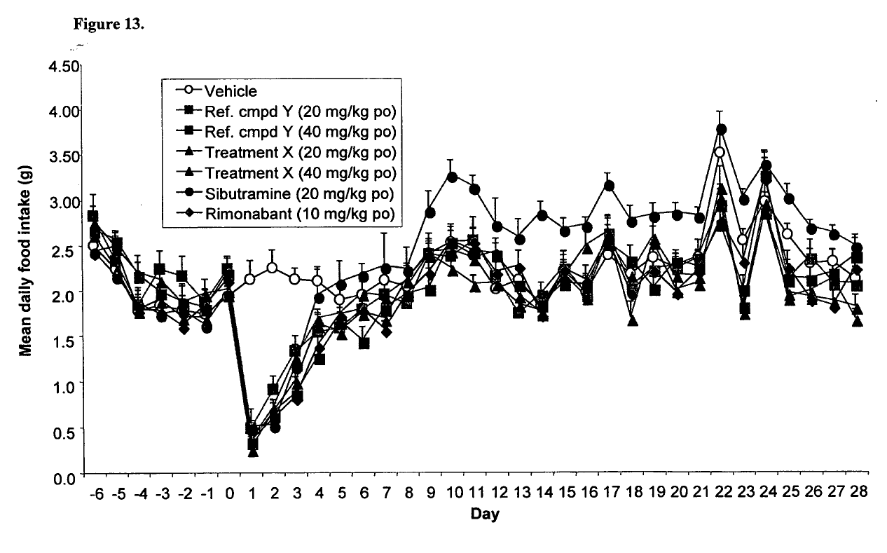

FIG. 13 shows the results of oral administration of Treatment X, Sibutramine and Rimonabant on the food intake of diet-induced obese male C57BL/6J mice. Drug treatment commenced on Day 1. Treatment X dosed orally at 20 mg/kg and 40 mg/kg demonstrated a similar food intake curve to Rimonabant dosed orally at 10 mg/kg.

All of the above-cited references and publications are hereby incorporated by reference.

### EQUIVALENTS

Those skilled in the art will recognize, or be able to ascertain using no more than routine experimentation, numerous equivalents to the specific method and reagents described herein. Such equivalents are considered to be within the scope of this invention and are covered by the following claims.

What is claimed is:

1. A method of treating obesity, an eating disorder, or sexual dysfunction in a mammalian host, comprising administering to a mammalian host suffering from obesity, an eating disorder, or sexual dysfunction an effective dose of a compound represented by any one of structural formulae I to IV or a pharmaceutically acceptable salt thereof, or a solvate or prodrug of the compound or its salt: 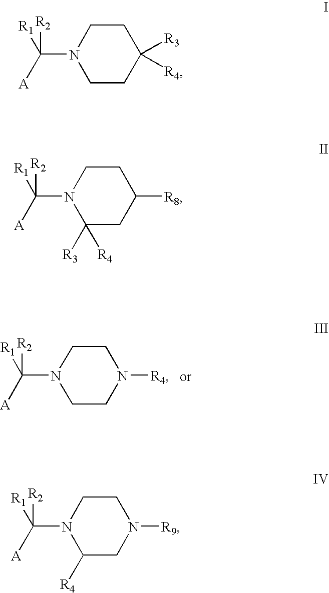
wherein:
R₁ and R₂ are each H or taken together are ═O;
R₃ is selected from OH, halogen, acyl, or substituted or unsubstituted C₁₋₆alkyl;
R₄ is selected from substituted or unsubstituted phenyl, substituted or unsubstituted thiophene, or ring B, wherein B is 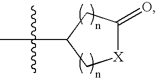
and ring B comprises 5, 6, or 7 atoms in the ring;
X is selected from O or NR₁₀;
R₁₀ is selected from H or substituted or unsubstituted C₁₋₆alkyl;
n is independently for each occurrence selected from 0, 1, 2, 3, or 4;
R₈ is selected from H, OH or substituted or unsubstituted C₁₋₆alkyl;
R₉ is selected from H or substituted or unsubstituted C₁₋₆alkyl;
A is selected from 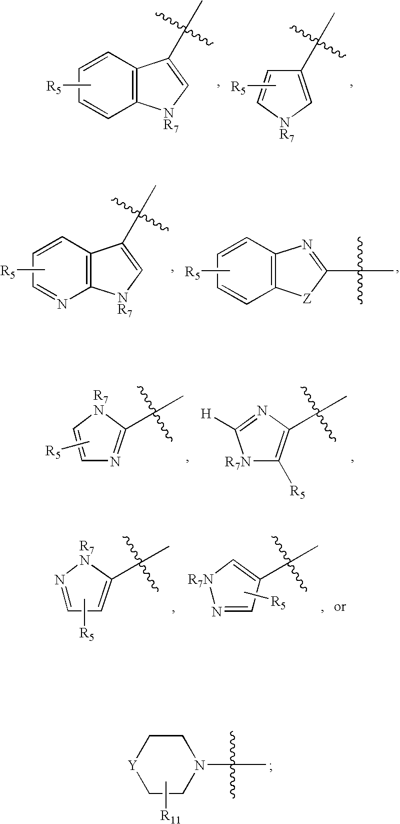
R₅ is independently for each occurrence selected from H, substituted or unsubstituted alkyl, alkenyl, alkynyl, aryl, aralkyl, alkoxy, aryloxy, aralkoxy, cycloalkyl, cycloalkoxy, heterocyclyl, heterocyclyloxy, heterocyclylalkyl, heteroaryl, or heteroaralkyl, keto, hydroxy, substituted or unsubstituted alkylthio, amino, alkylamino, alkanoylamino, aroylamino, or aralkanoylamino, carboxy, substituted or unsubstituted carboxyalkyl, carboxamidoalkyl, thiocarboxy, thiocarboxyalkyl, or thiocarboxamidoalkyl, halo, cyano, nitro, formyl, acyl, or substituted or unsubstituted sulfonyl, or sulfonamido, and is optionally substituted with 1-3 R₆ groups;
R₁₁ is independently for each occurrence selected from H, substituted or unsubstituted alkyl, alkenyl, alkynyl, aryl, aralkyl, alkoxy, aryloxy, aralkoxy, cycloalkyl, cycloalkoxy, heterocyclyl, heterocyclyloxy, heterocyclylalkyl, heteroaryl, or heteroaralkyl, keto, hydroxy, substituted or unsubstituted alkylthio, amino, alkylamino, alkanoylamino, aroylamino, or aralkanoylamino, carboxy, substituted or unsubstituted carboxyalkyl, carboxamidoalkyl, thiocarboxy, thiocarboxyalkyl, or thiocarboxamidoalkyl, halo, cyano, nitro, formyl, acyl, or substituted or unsubstituted sulfonyl, or sulfonamido, and is optionally substituted with 1-3 R₆ groups;
R₆ is independently for each occurrence selected from substituted or unsubstituted alkyl, aryl, aralkyl, alkoxy, aryloxy, heterocyclyl, or heterocyclyloxy, keto, hydroxy, substituted or unsubstituted alkylthio, amino, alkanoylamino, or aroylamino, carboxy, substituted or unsubstituted carboxyalkyl, or carboxamidoalkyl, halo, cyano, nitro, formyl, or substituted or unsubstituted sulfonyl, or sulfonamido;
Y is CH₂, O, S or NR₁₀;
Z is selected from O or NR₇; and
R₇ is independently for each occurrence selected from H or substituted or unsubstituted C₁₋₆alkyl;
wherein when the compound is represented by formula III and R₁ and R₂ are each H, then R₅ and R₇ are each H.

2. The method of claim 1 characterized by one or more of the following: 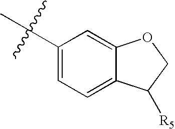
R₄ is 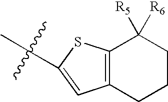
and R₅ is defined as in claim 1;
wherein the compound is optionally enriched in one of the stereoisomers at the site of attachment of R₅ to R₄;
R₄ is
and R₅ and R₆ are defined as in claim 1;
wherein the compound is optionally enriched in one of the stereoisomers at the site of attachment of R₅ and R₆ to R₄;
the compound is represented by structural formula II and is enriched in one of the stereoisomers at the site of attachment of R₃ and R₄;
R₃ is selected from OH, F, C(O)CF₃, or CH₃;
R₃ is OH;
R₈ is selected from H, OH or CH₃;
R₉ is selected from H or CH₃;
the compound is 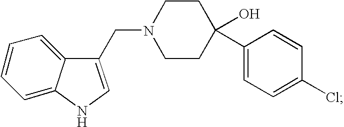
the compound is 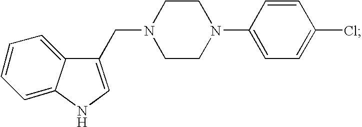
the effective dose is in the range of 5 mg/day and 30 mg/day;
the compound is administered as a pharmaceutical composition comprising a pharmaceutically acceptable carrier;
the mammalian host is a patient being treated with one or more anti-psychotic agents;
wherein the anti-psychotic agent is optionally an atypical anti-psychotic agent;
further comprising administering to the mammalian host an antagonist of the CB1 receptor;
wherein the antagonist of the CB1 receptor is optionally norfluoxetine enriched for the (R) enantiomer;
further comprising administering to the mammalian host an agonist of the MC4 receptor;
further comprising administering to the mammalian host an inhibitor of dopamine reuptake;
further comprising administering to the mammalian host an inhibitor of norepinephrine reuptake;
further comprising administering to the mammalian host an inhibitor of both dopamine and norepinephrine reuptake;
further comprising administering to the mammalian host a dopamine agonist or partial agonist;
further comprising administering to the mammalian host bupropion, methylphenidate, sibutramine, sertraline, venlafaxine, atomoxetine, amineptine, benztropine, reboxetine, or a metabolite or stereoisomer thereof; or
the mammalian host is a human.

3-25. (canceled)

26. A method of potentiating the effect of an MC4 receptor agonist in a mammalian host, comprising administering to the mammalian host a compound that lowers the EC₅₀ of the agonist for the MC4 receptor by at least 10%, at least 20%, at least 30%, at least 40%, at least 50%, at least 60%, at least 70%, or at least 80%.

27. A method of potentiating the effect of an MC4 receptor agonist in a mammalian host, comprising administering to the mammalian host a compound that increases the maximum effect of the agonist on the MC4 receptor by at least 10%, at least 20%, at least 30%, at least 40%, at least 50%, at least 60%, at least 70%, or at least 80%.

28. The method of claim 26 or 27 characterized by one or more of the following:
the compound is an allosteric potentiator of an MC4 receptor agonist;
the agonist is α-MSH or NDP α-MSH;
the compound further attenuates the effect of an MC4 receptor inverse agonist in a mammalian host and raises the EC₅₀ of the inverse agonist for the MC4 receptor by at least 10%, at least 20%, at least 30%, at least 40%, at least 50%, at least 60%, at least 70%, or at least 80%;
the compound further attenuates the effect of an MC4 receptor inverse agonist in a mammalian host and decreases the maximum effect of the inverse agonist on the MC4 receptor by at least 10%, at least 20%, at least 30%, at least 40%, at least 50%, at least 60%, at least 70%, or at least 80%;
wherein the MC4 receptor inverse agonist is optionally AgRP,
the compound is an allosteric attenuator of an MC4 receptor inverse agonist;
wherein the MC4 receptor inverse agonist is optionally AgRP:
the mammalian host suffers from obesity, an eating disorder, or sexual dysfunction, and the compound is administered in an effective dose;
the compound is represented by any one of structural formulae I to IV or a pharmaceutically acceptable salt thereof, or a solvate or prodrug of the compound or its salt: 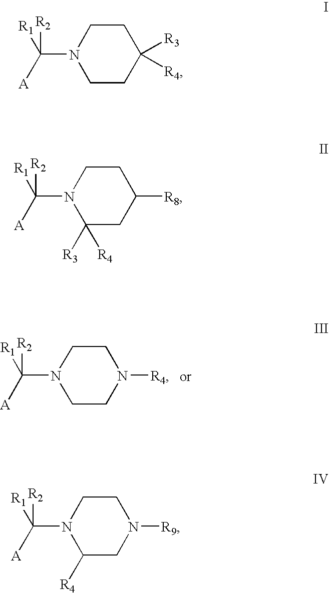
wherein:
R₁ and R₂ are each H or taken together are ═O;
R₃ is selected from OH, halogen, acyl, or substituted or unsubstituted C₁₋₆alkyl:
R₄ is selected from substituted or unsubstituted phenyl, substituted or unsubstituted thiophene, or ring B, wherein B is 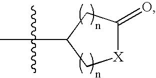
and ring B comprises 5, 6, or 7 atoms in the ring;
X is selected from O or NR₁₀;
R₁₀ is selected from H or substituted or unsubstituted C₁₋₆alkyl;
n is independently for each occurrence selected from 0.1, 2, 3, or 4;
R₈ is selected from H, OH or substituted or unsubstituted C₁₋₆alkyl:
R₉ is selected from H or substituted or unsubstituted C₁₋₆alkyl;
A is selected from 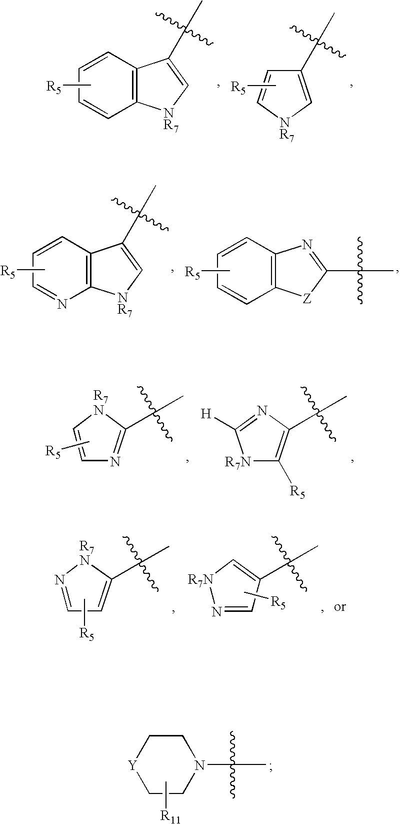
R₅ is independently for each occurrence selected from H, substituted or unsubstituted alkyl, alkenyl, alkynyl, aryl, aralkyl, alkoxy, aryloxy, aralkoxy, cycloalkyl, cycloalkoxy, heterocyclyl, heterocyclyloxy, heterocyclylalkyl, heteroaryl, or heteroaralkyl, keto, hydroxy, substituted or unsubstituted alkylthio, amino, alkylamino, alkanoylamino, aroylamino, or aralkanoylamino, carboxy, substituted or unsubstituted carboxyalkyl, carboxamidoalkyl, thiocarboxy, thiocarboxyalkyl, or thiocarboxamidoalkyl, halo, cyano, nitro, formyl, acyl, or substituted or unsubstituted sulfonyl, or sulfonamido, and is optionally substituted with 1-3 R₆ groups;
R₁₁ is independently for each occurrence selected from H, substituted or unsubstituted alkyl, alkenyl, alkynyl, aryl, aralkyl, alkoxy, aryloxy, aralkoxy, cycloalkyl, cycloalkoxy, heterocyclyl, heterocyclyloxy, heterocyclylalkyl, heteroaryl, or heteroaralkyl, keto, hydroxy, substituted or unsubstituted alkylthio, amino, alkylamino, alkanoylamino, aroylamino, or aralkanoylamino, carboxy, substituted or unsubstituted carboxyalkyl, carboxamidoalkyl, thiocarboxy, thiocarboxyalkyl, or thiocarboxamidoalkyl, halo, cyano, nitro, formyl, acyl, or substituted or unsubstituted sulfonyl, or sulfonamido, and is optionally substituted with 1-3 R₆ groups;
R₆ is independently for each occurrence selected from substituted or unsubstituted alkyl, aryl, aralkyl, alkoxy, aryloxy, heterocyclyl, or heterocyclyloxy, keto, hydroxy, substituted or unsubstituted alkylthio, amino, alkanoylamino, or aroylamino, carboxy, substituted or unsubstituted carboxyalkyl, or carboxamidoalkyl, halo, cyano, nitro, formyl, or substituted or unsubstituted sulfonyl, or sulfonamido;
Y is CH₂, O, S or NR₁₀;
Z is selected from O or NR₇ and
R₇ is independently for each occurrence selected from H or substituted or unsubstituted C₁₋₆alkyl;
wherein when the compound is represented by formula III and R₁ and R₂ are each H, then R₅ and R₇ are each H;
wherein the method is optionally characterized by one or more of the following:
R₄ is 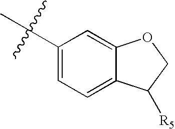
and R₅ is defined as in claim 38:
wherein the compound is enriched in one of the stereoisomers at the site of attachment of R₅ to R₄;
R₄ is 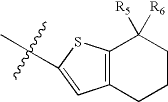
and R₅ and R₆ are defined as in claim 38:
wherein the compound is enriched in one of the stereoisomers at the site of attachment of R₅ and R₆ to R₄;
the compound is represented by structural formula II and is enriched in one of the stereoisomers at the site of attachment of R₃ and R₄;
R₃ is selected from OH, F, C(O)CF, or CH₃;
R₃ is OH;
R⁸ is selected from H, OH or CH₃;
R₉ is selected from H or CH₃;
the compound is 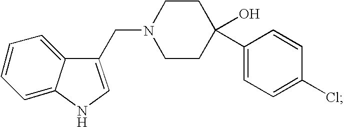
the compound is 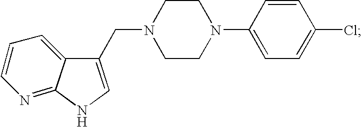
the effective dose is in the range of 5 mg/day and 30 mg/day;
the compound is administered as a pharmaceutical composition comprising a pharmaceutically acceptable carrier;
the mammalian host is a patient being treated with one or more anti-psychotic agents,
wherein the anti-psychotic agent is optionally an atypical anti-psychotic agent;
further comprising administering to the mammalian host an antagonist of the CB1 receptor;
wherein the antagonist of the CB1 receptor is optionally norfluoxetine enriched for the (R) enantiomer;
further comprising administering to the mammalian host an agonist of the MC4 receptor;
further comprising administering to the mammalian host an inhibitor of dopamine reuptake;
further comprising administering to the mammalian host an inhibitor of norepinephrine reuptake;
further comprising administering to the mammalian host an inhibitor of both dopamine and norepinephrine reuptake;
further comprising administering to the mammalian host a dopamine agonist or partial agonist;
further comprising administering to the mammalian host bupropion, methylphenidate, sibutramine, sertraline, venlafaxine, atomoxetine, amineptine, benztropine, reboxetine, or a metabolite or stereoisomer thereof, or
the mammalian host is a human.

29-34. (canceled)

35. A method of allosterically attenuating the effect of an MC4 receptor inverse agonist in a mammalian host, comprising administering to the mammalian host a compound that raises the EC₅₀ of the inverse agonist for the MC4 receptor by at least 10%, at least 20%, at least 30%, at least 40%, at least 50%, at least 60%, at least 70%, or at least 80%.

36. A method of allosterically attenuating the effect of an MC4 receptor inverse agonist in a mammalian host, comprising administering to the mammalian host a compound that decreases the maximum effect of the inverse agonist on the MC4 receptor by at least 10%, at least 20%, at least 30%, at least 40%, at least 50%, at least 60%, at least 70%, or at least 80%.

37. The method of claim 35 or 36 characterized by one or more of the following:
the MC4 receptor inverse agonist is AgRP;
the compound is represented by any one of structural formulae I to IV or a pharmaceutically acceptable salt thereof, or a solvate or prodrug of the compound or its salt: 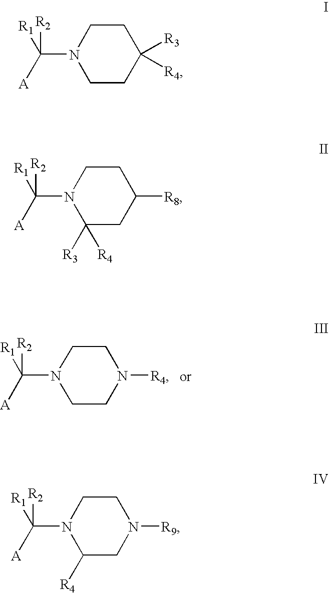
wherein:
R₁ and R₂ are each H or taken together are ═O;
R₃ is selected from OH, halogen, acyl, or substituted or unsubstituted C₁₋₆alkyl;
R₄ is selected from substituted or unsubstituted phenyl, substituted or unsubstituted thiophene, or ring B, wherein B is 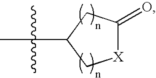
and ring B comprises 5, 6, or 7 atoms in the ring;
X is selected from O or NR₁₀;
R₁₀ is selected from H or substituted or unsubstituted C₁₋₆alkyl;
n is independently for each occurrence selected from 0, 1, 2, 3, or 4;
R₈ is selected from H, OH or substituted or unsubstituted C₁₋₆alkyl;
R₉ is selected from H or substituted or unsubstituted C₁₋₆alkyl;
A is selected from 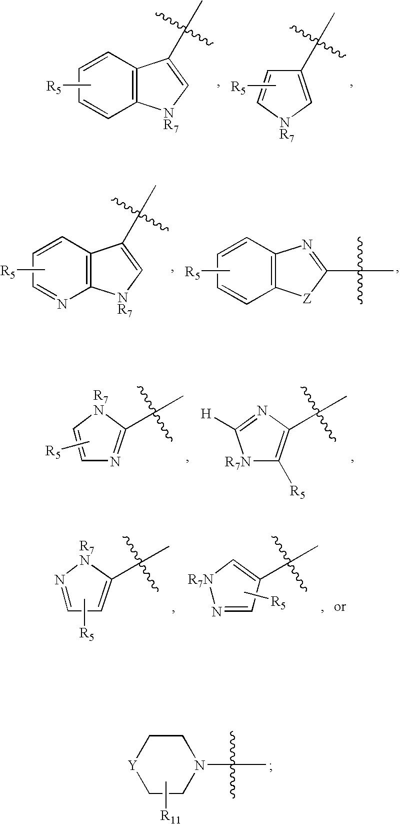
R₅ is independently for each occurrence selected from H, substituted or unsubstituted alkyl, alkenyl, alkynyl, aryl, aralkyl, alkoxy aryloxy, aralkoxy, cycloalkyl, cycloalkoxy, heterocyclyl, heterocyclyloxy, heterocyclylalkyl, heteroaryl, or heteroaralkyl, keto, hydroxy, substituted or unsubstituted alkylthio, amino, alkylamino, alkanoylamino, aroylamino, or aralkanoylamino, carboxy, substituted or unsubstituted carboxyalkyl, carboxamidoalkyl, thiocarboxy, thiocarboxyalkyl, or thiocarboxamidoalkyl, halo, cyano, nitro, formyl, acyl, or substituted or unsubstituted sulfonyl, or sulfonamido, and is optionally substituted with 1-3 R₆ groups;
R₁₁ is independently for each occurrence selected from H, substituted or unsubstituted alkyl, alkenyl, alkynyl, aryl, aralkyl, alkoxy, aryloxy, aralkoxy, cycloalkyl, cycloalkoxy, heterocyclyl, heterocyclyloxy, heterocyclylalkyl, heteroaryl, or heteroaralkyl, keto, hydroxy, substituted or unsubstituted alkylthio, amino, alkylamino, alkanoylamino, aroylamino, or aralkanoylamino, carboxy, substituted or unsubstituted carboxyalkyl, carboxamidoalkyl, thiocarboxy, thiocarboxyalkyl, or thiocarboxamidoalkyl, halo, cyano, nitro, formyl, acyl, or substituted or unsubstituted sulfonyl, or sulfonamido, and is optionally substituted with 1-3 R₆ groups,
R₆ is independently for each occurrence selected from substituted or unsubstituted alkyl, aryl, aralkyl, alkoxy, aryloxy, heterocyclyl, or heterocyclyloxy, keto, hydroxy, substituted or unsubstituted alkylthio, amino, alkanoylamino, or aroylamino, carboxy, substituted or unsubstituted carboxyalkyl, or carboxamidoalkyl, halo, cyano, nitro, formyl, or substituted or unsubstituted sulfonyl, or sulfonamido;
Y is CH₂, O, S or NR₁₀;
Z is selected from O or NR₇; and
R₇ is independently for each occurrence selected from H or substituted or unsubstituted C₁₋₆alkyl;
wherein when the compound is represented by formula III and R₁ and R₂ are each H, then R₅ and R₇ are each H;
wherein the compound is optionally characterized by one or more of the following:
R₄ is 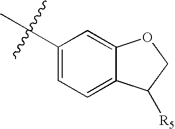
and R₅ is defined as in claim 38;
wherein the compound is enriched in one of the stereoisomers at the site of attachment of R₃ to R₄;
R₄ is 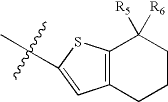
and R₅ and R₆ are defined as in claim 38;
wherein the compound is enriched in one of the stereoisomers at the site of attachment of R₅ and R₆ to R₄;
the compound is represented by structural formula II and is enriched in one of the stereoisomers at the site of attachment of R₃ and R₄;
R₃ is selected from OH, F, C(O)CF₃, or CH₃;
R₃ is OH;
R₈ is selected from H, OH or CH₃;
R₉ is selected from H or CH₃;
the compound is 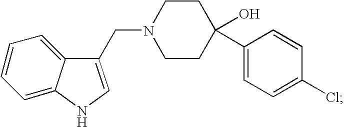
the compound is 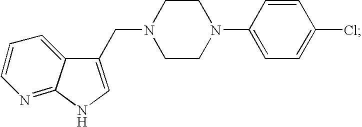
the effective dose of the compound is in the range of 5 mg/day and 30 mg/day;
the compound is administered as a pharmaceutical composition comprising a pharmaceutically acceptable carrier;
the mammalian host is a patient being treated with one or more anti-psychotic agents;
wherein the anti-psychotic agent is optionally an atypical anti-psychotic agent;
further comprising administering to the mammalian host an antagonist of the CB1 receptors
wherein the antagonist of the CB1 receptor is optionally norfluoxetine enriched for the (R) enantiomer,
further comprising administering to the mammalian host an agonist of the MC4 receptor;
further comprising administering to the mammalian host an inhibitor of dopamine reuptake;
further comprising administering to the mammalian host an inhibitor of norepinephrine reuptake;
further comprising administering to the mammalian host an inhibitor of both dopamine and norepinephrine reuptake;
further comprising administering to the mammalian host a dopamine agonist or partial agonist;
further comprising administering to the mammalian host bupropion, methylphenidate, sibutramine, sertraline, venlafaxine, atomoxetine, amineptine, benztropine, reboxetine, or a metabolite or stereoisomer thereof; or
the mammalian host is a human.

38-112. (canceled)
